(19) United States
(12) Patent Application Publication
(10) Pub. No.: US 2023/0053307 A1
(43) Pub. Date: Feb. 16, 2023

(54) METHOD FOR PREVENTING OR TREATING SKIN DISORDERS AND CONDITIONS

(71) Applicant: BUDDHIST TZU CHI MEDICAL FOUNDATION, Hualien (TW)

(72) Inventor: Chung-Hsing Chang, Hualien (TW)

(21) Appl. No.: 17/378,839

(22) Filed: Jul. 19, 2021

(51) Int. Cl. A61K 31/506; A61K 31/472; A61K 31/4439; A61K 31/404; A61P 17/16

(52) U.S. Cl. CPC A61K 31/506; A61K 31/472; A61K 31/4439; A61K 31/404; A61P 17/16

(57) ABSTRACT

Provided are methods for preventing, ameliorating, or treating a skin disorder, disease, or condition with a casein kinase 1 inhibitor. Also provided are methods of increasing skin pigmentation or sunburn protection by inhibiting casein kinase 1.

### BACKGROUND

### 1. Technical Field

The present disclosure relates to methods for preventing, ameliorating or treating a skin disorder, disease, or condition with a casein kinase 1 inhibitor. Also provided herein are methods of increasing skin pigmentation by inhibiting casein kinase 1.

### 2. Description of Related Art

Ultraviolet (UV) can injure the skin both by indirect cellular damage via the generation of reactive oxygen species (ROS) and by direct damage to the nucleotide structure in DNA, thereby causing an acute sunburn reaction. Therefore, UV radiation is highly related to development of skin cancers, among other skin disorders and conditions.

Epidermis is mainly composed of keratinocytes and melanocytes and acts as a highly sophisticated barrier tissue that protects the body against continuous external injuries such as UV radiation. Keratinocytes are sensitive to UV and are the major responders in the skin. They produce various paracrine factors in response to UV, which influence their microenvironment and activate adjacent melanocytes, forming a keratinocyte-melanocyte functional unit. Skin hyperpigmentation, which results from the increased synthesis of melanin in melanocytes followed by the distribution of melanin to neighboring keratinocytes, is one of the biological reactions of the body against UV.

Melanin therefore acts as a natural sunscreen that directly protects against UV and visible light radiation from penetrating to deep skin layers where proliferating cells reside, and acts as a potent antioxidant and free-radical scavenger. Individuals with darker skin have a reduced incidence of UV-induced skin cancers, whereas individuals with lighter skin are more prone to UV-induced damage and tumor formation and have weak tanning responses. In fact, melanocytes produce two distinct types of melanin pigments, which are black-brown eumelanin and yellow-reddish pheomelanin.

Although both melanin pigments can be found in populations with different skin colors, the black-brown eumelanin is found in a dominant amount in individuals with black and/or brown hair, while the yellow-reddish pheomelanin is primarily produced in individuals with red hair and freckles. However, the beneficial effects of melanin mainly result from the presence of eumelanin that absorbs most of the UV and scavenges the UV-generated free radicals, whereas pheomelanin is known to be carcinogenic. Therefore, manipulation of melanin pigments should be carefully implemented to selectively increase the level of eumelanin but not that of pheomelanin.

Therefore, an effective method to selectively increase the beneficial level of eumelanin is highly sought after for prevention of UV-induced DNA damage and skin cancers.

### SUMMARY

Provided herein is a method for preventing, ameliorating, or treating a skin disorder, disease, or condition in a subject in need thereof, comprising administering to the subject an effective amount of a casein kinase 1 inhibitor (CKI). In at least one embodiment, the skin disorder, disease, or condition is a sunburn. In at least one embodiment, the skin disorder, disease, or condition is an acute sunburn. In at least one embodiment, the skin disorder, disease, or condition is hypopigmentation. In some embodiments, the skin disorder, disease, or condition is caused by a defect in the signaling pathway involving at least one of pro-opiomelanocortin (POMC), a-melanocyte stimulating hormone (a-MSH), melanocortin 1 receptor (MC1R) and microphthalmia-associated transcription factor (MITF).

Also provided herein is a method for protecting a subject from ultraviolet radiation, comprising administering to the subject an effective amount of a casein kinase 1 inhibitor.

Future provided herein is a method for increasing skin pigmentation in a subject in need thereof, comprising administering to the subject an effective amount of a casein kinase 1 inhibitor.

Provided herein is a method for increasing a eumelanin level in a subject in need thereof, comprising administering to the subject an effective amount of a casein kinase 1 inhibitor. In some embodiments, increasing the eumelanin level involves an increase in a KitL level. In some embodiments, increasing the KitL level in the epidermis induces movement of melanocytes from dermis to the epidermis in the subject. In at least one embodiment, the eumelanin level is increased selectively over the pheomelanin level. In some embodiments, the eumelanin level is increased in epidermal of the subject.

In at least one embodiment, the methods provided herein involve increase of melanocyte cell number or migration of melanocytes from dermis to epidermis.

In at least one embodiment, the methods provided herein involve topical administration of a casein kinase 1 inhibitor.

Provided herein is a method of inhibiting the activity of a casein kinase 1 in a skin cell, comprising contacting the skin cell with an effective amount of a casein kinase 1 inhibitor.

Provided herein is a method of increasing a eumelanin level in a skin cell, comprising contacting the skin cell with an effective amount of a casein kinase 1 inhibitor.

### BRIEF DESCRIPTION OF THE DRAWINGS

The present disclosure will become more readily appreciated by reference to the following descriptions in conjunction with the accompanying drawings.

FIGS. 1A to 1H illustrate the UV protection effect of topical treatment of a CKI, A51, on wild-type (WT) mice. FIG. 1A shows the treatment scheme of topical application of CKI on C57BL/6 mice ears every other day for four weeks at the amount of 0.1 mg/cm² for each application with a total of 1.2 mg CKI applied. Arrow at week 6 indicates the harvest time point of ear samples for tissue analysis (N=10). FIG. 1B shows the photos of ear phenotypes, Fontana-Masson staining, c-Kit staining and Trp1 staining by immunohistochemistry (IHC) analysis of ear tissues. Also shown are epidermal thickness, melanin intensity, and number of c-Kit* and Trp1+cells quantitated from the staining and represented in histograms. The detailed skin pigmentation was taken by digital microscope (UPG650, Upmost Technology Co.).

Increased melanocyte number was observed in epidermis at week 6. Fontana-Masson staining demonstrated an increased eumelanin amount in epidermis and increased epidermal thickness. c-Kit staining showed increased Kit expression on keratinocytes and melanocytes. Trpl staining showed increased melanocyte number in epidermis.

FIG. 1C shows the significantly increased eumelanin (EM) to pheomelanin (PM) ratio in the whole skin after topical CKI application, and the increased eumelanin ratio was mainly localized in the epidermis rather than in the dermis. NS: not significant. FIGS. 1D and 1E show the result of UV protection effect on wild-type mice (Control) with a total of 5 mg CKI treatment over 4 weeks following the scheme of 1 mg/cm² once, followed by 0.5 mg/cm² for 6 times and 0.2 mg/cm² for 5 times, topically applied to the mice ears every other day. In FIG. 1F, the Tyr-CreER^{T2}; FusionRed mice are generated and used for tracing melanocyte by FusionRed fluorescence from Cre-recombination driven by melanocyte-specific tyrosinase promoter. After tamoxifen induction, melanocytes specifically express red fluorescence (Fusion Red) on their membrane among the green fluorescence (GFP) cells in the tissue. Ear skins were harvested after tamoxifen induction and trimmed into 15 m sections. The sections were mounted under DAPI-containing mounting solution and observed under fluorescence microscope. In control mice, there is rare FusionRed-labelled melanocytes on ear epidermis (N=3). After topical application of 1.2 mg CKI on mouse ear, FusionRed-labelled melanocytes in epidermis, indicated by arrows, are increased (N=3). Number of melanocytes in epidermis were counted and shown in the histograms. FIG. 1G shows the protein expression levels in the KitL/c-Kit pathway and melanogenesis related targets by Western blotting analysis of the dorsal ear skin tissues. Light gray bars represent the Control group, while dark gray bars represent the group with 1.2 mg CKI treatment. n.s.: not significant. FIG. 1H shows the treatment scheme and results of the protection effect of topical CKI treatment on UVB irradiation-induced short-term keratinocyte DNA damage. After a total of 0.6 mg CKI treatment with topical application of 0.1 mg CKI every other day for a total of 6 applications, mice were exposed to 750 mJ/cm² UVB (N=6) for 24 hours on day 21. The harvested samples were fixed in 4% paraformaldehyde (PFA) and embedded in paraffin or frozen optimal cutting temperature (OCT). The DNA damage caused by UVB exposure was examined by the amount of cyclobutane pyrimidine dimers (CPDs), the specific marker of the impingement of UVB radiation. Reduced CPD staining after CKI treatment shows that topical CKI's UV protection effect is effective at a lower CKI dose (0.6 mg), before the visible darker skin treated with higher CKI dose (1.2 mg or 5 mg). The signals were all quantified by ImageJ software, and the statistical results were determined by unpaired t-test analysis and showed as mean±SD. The asterisks *** indicated a significant difference with P<0.001.

FIGS. 2A to 2L show the effects of CK1α ablation in keratinocytes of MC1R mutant mice and the UV protection effect of topical CKI treatment on MC1R mutant mice. FIG. 2A shows the phenotypes of C57BL/6 and C57BL/6J-Mc1r^{em1} mice and the generation of CK1α ablation in keratinocytes of C57BL/6J-Mc1r^{em1} mice, and FIG. 2B shows the scheme of mice crossing to obtain an inducible MC1R mutant mice. FIG. 2C shows the scheme for tamoxifen induction and the skin sample harvest on days 14, 28 and 42. FIG. 2D shows the phenotype of skin in MC1R mutant mice with CK1α ablation in keratinocytes. FIG. 2E shows the Fontana-Masson staining of skin of MC1R mutant mice with CK1α ablation in keratinocytes. FIG. 2F shows the quantitated number of Fontana-Masson-stained cells in the whole skin (Whole), epidermis and dermis, demonstrating an increase of eumelanin in epidermis, dermis and whole skin. FIG. 2G shows the treatment scheme of topical CKI application, where CKI is topically applied for 4 weeks at 0.1 mg/time and 0.3 mg/week with a total of 1.2 mg CKI applied and skin harvested at the₆th week for tissue analysis (N=6). FIG. 2H shows the ear samples harvested 6 weeks after topical CKI treatment subjected to Fontana-Masson staining for observation of epidermal thickness and eumelanin amount, respectively. FIG. 2I shows the expression levels of proteins in the KitL/c-Kit pathway and melanocyte-related targets in the ear sample by Western blotting analysis, indicating that expressions of KitL, c-Kit, MITF, tyrosinase and p53 were all significantly increased after topical treatment of 1.2 mg CKI in C57BL/6J-Mc1r^{em1} mice, as compared to topically vehicle-treated mice. FIG. 2J shows an increased ratio of EM/PM in C57BL/6J-Mc1r^{em1} mice treated with topical application of a total of 2.6 mg CKI. FIG. 2K shows the effect of UVB radiation on C57BL/6J-Mc1r^{em1} mice at 750 mJ/cm² with or without CKI treatment. The skin samples were treated as illustrated in the scheme and harvested after 24 hours of 750 mJ/cm² UVB irradiation on day 21 and stained with anti-CPD antibody for examining the DNA damage. The mutant mice treated with a total of 0.6 mg CKI showed reduced cyclobutene pyrimidine dimer (CPD) staining in epidermis and dermis (sebaceous epithelium, hair follicle epithelium, fibroblast), indicating the UV protection effect of topical CKI treatment. The histogram shows the number of CPD-positive cells in epidermis and dermis of control and in CKI-treated group, respectively. FIG. 2L compares the CPD staining obtained from wild-type (WT) mice and MC1R mutant mice with or without topical CKI treatment and shows that topical CKI treatment prevents skin from UV damage in both WT and MC1R mutant mice.

FIGS. 3A to 3E show that topical CKI application exerts UV protection effect that prevents UVB-induced DNA damage in human skin explants. FIG. 3A shows the scheme of CKI treatment using human foreskin specimen. The specimen was cultured in the 12-well plate (N=5). After 24 hours of explant culture, the specimen was treated with 0.01 mg CKI, and another treatment of 0.01 mg CKI was given two days thereafter, as shown in the timeline. On the 5th day, the specimens were exposed under 1 J/cm² UVB light. Samples were harvested 6 hours later. Photos of the skin specimen showed darker skin after topical CKI treatment. FIG. 3B shows the increased ratio of EM/PM after topical CKI treatment with a total of 0.02 mg. FIG. 3C shows the photograph of the tissues for direct observation that were fixed with 4% PFA and then embedded with paraffin for melanin distribution by Fontana-Masson staining. Moreover, frozen embedding in OCT was also made for IHC analysis for Melan-A and Trp-1 labelling.

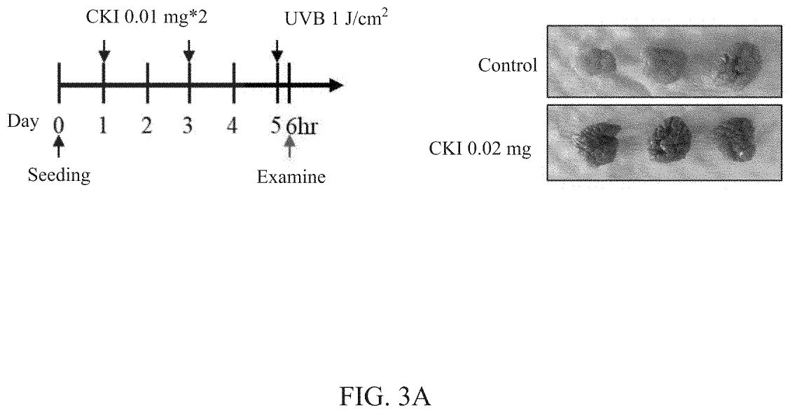

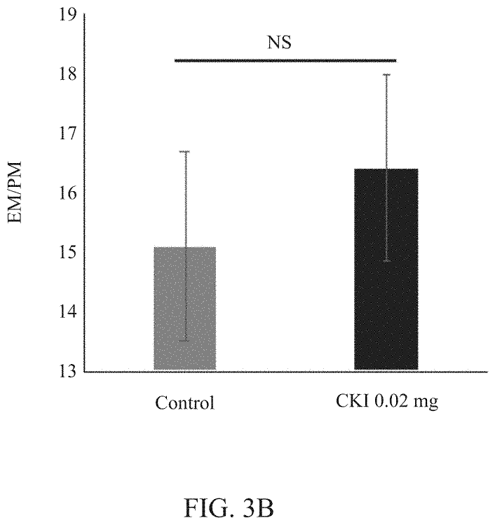

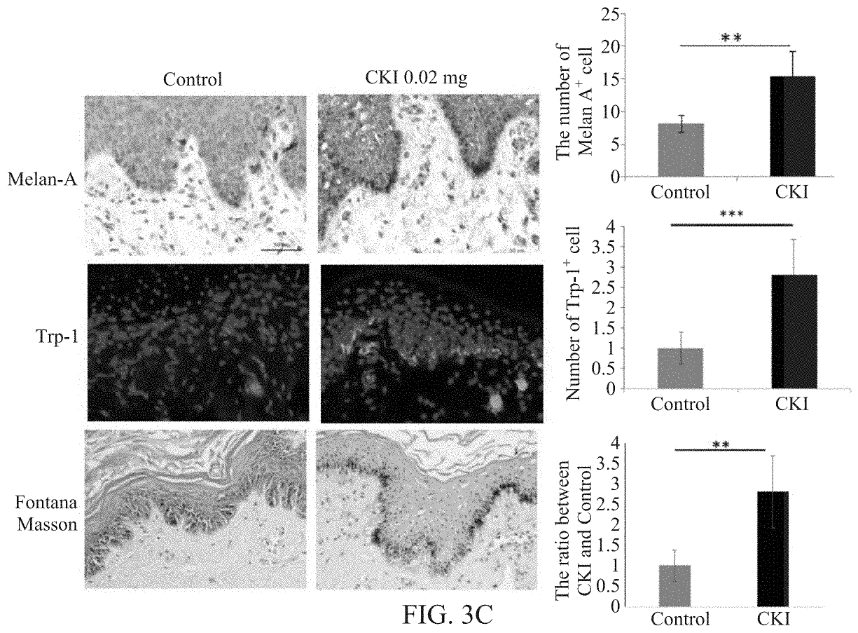

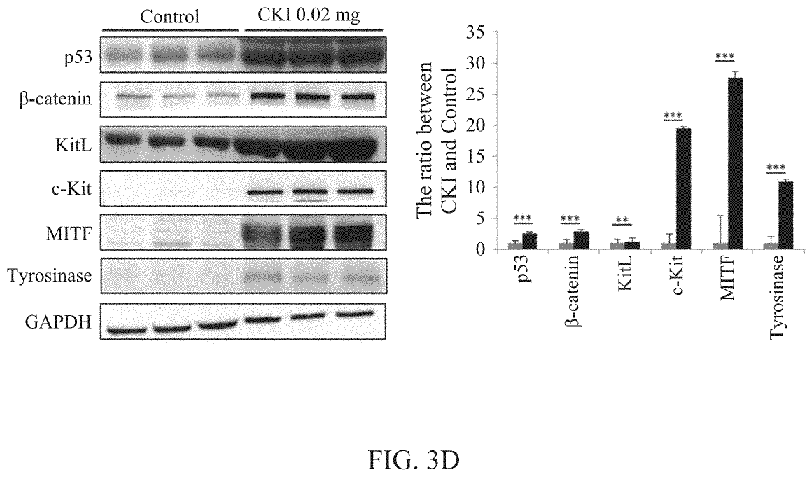

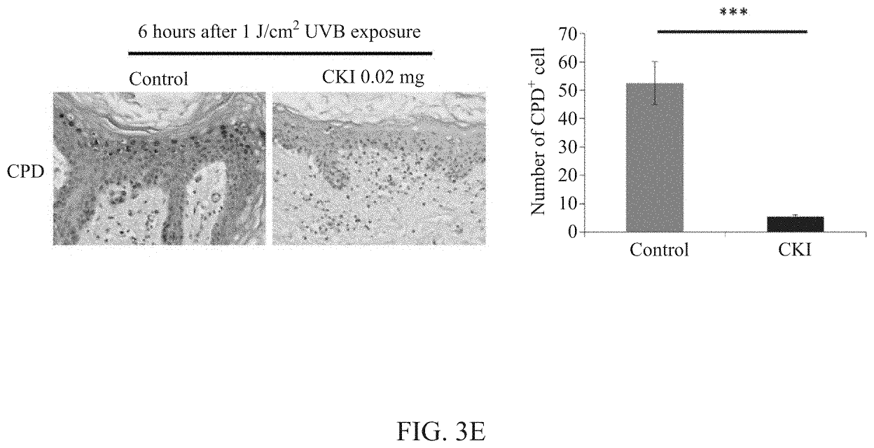

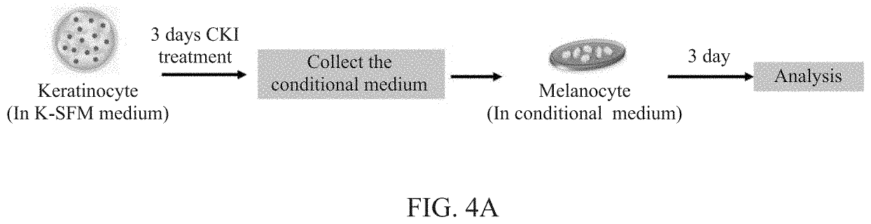

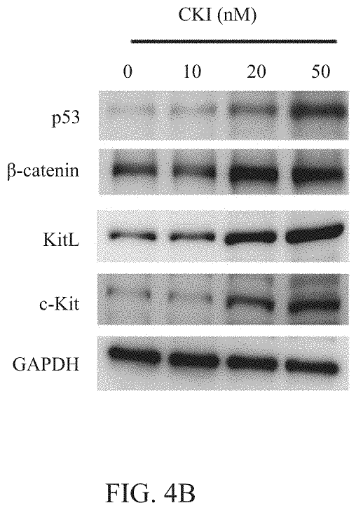

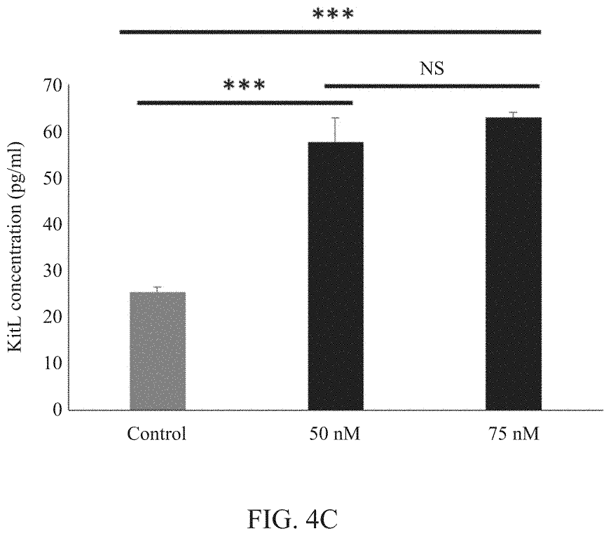

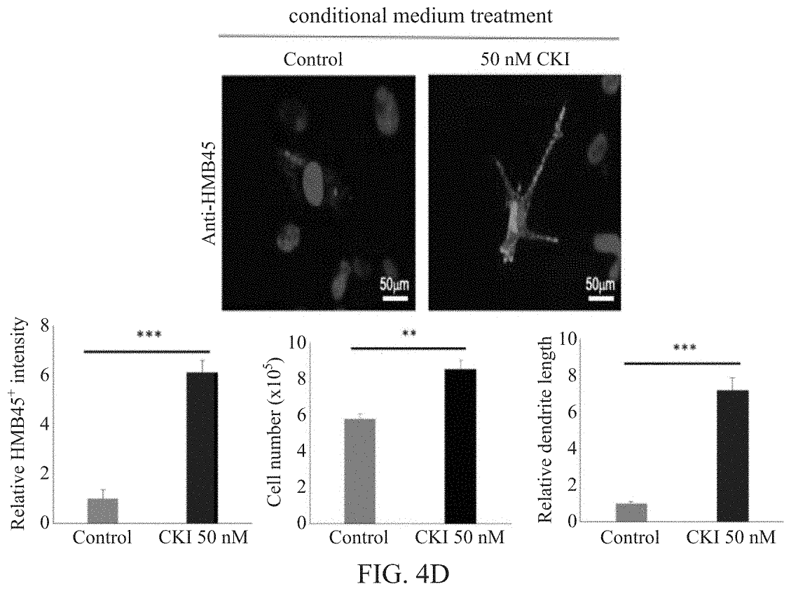

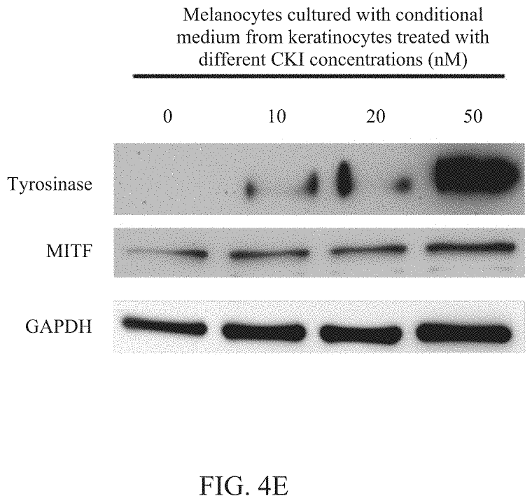

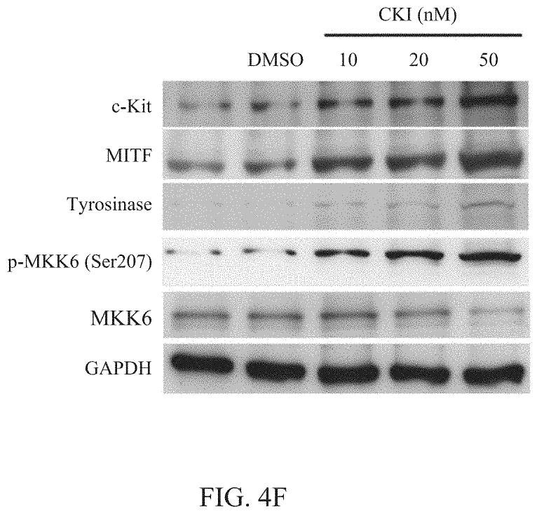

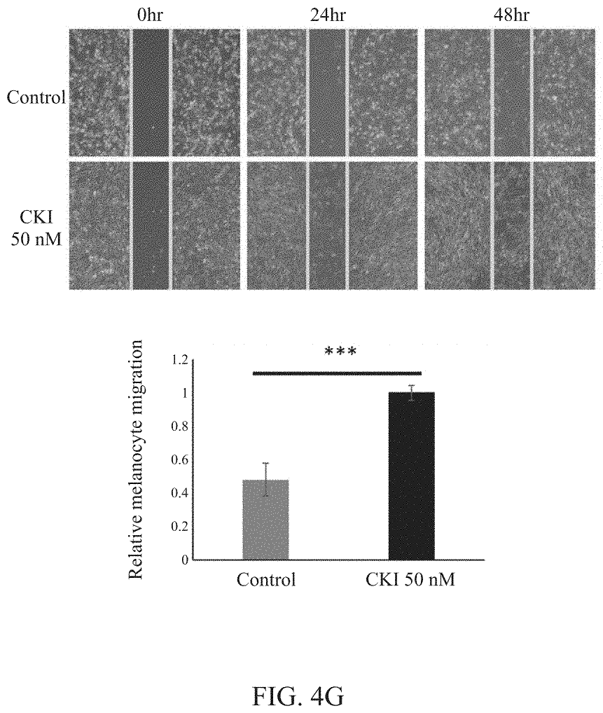

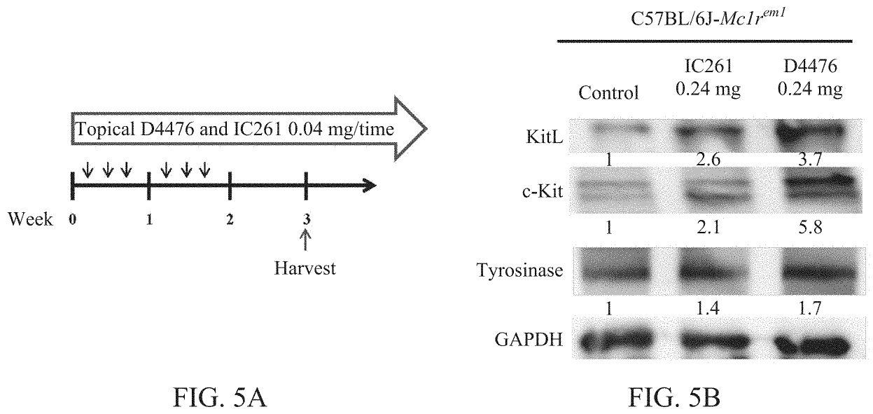

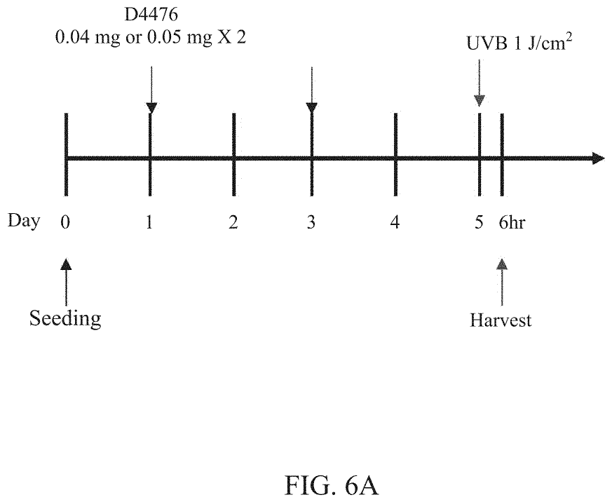

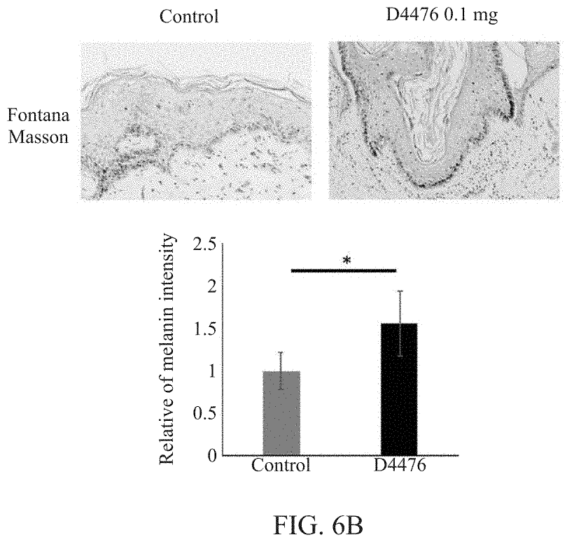

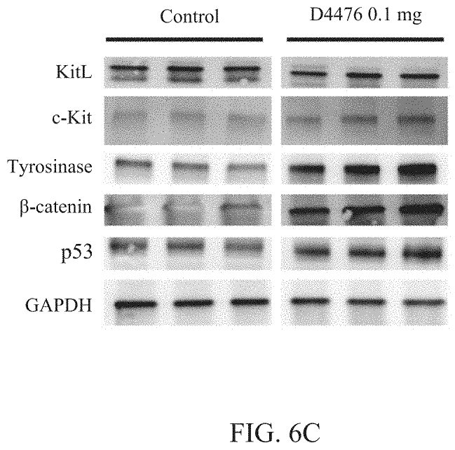

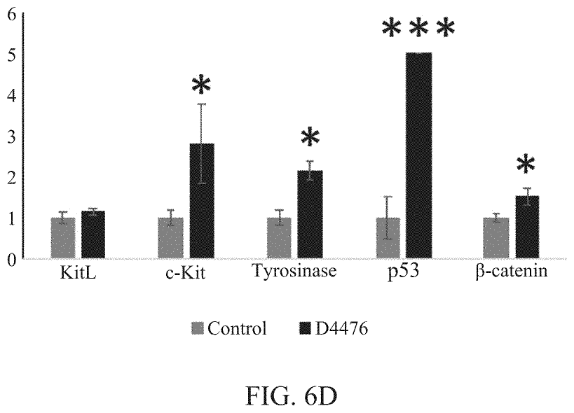

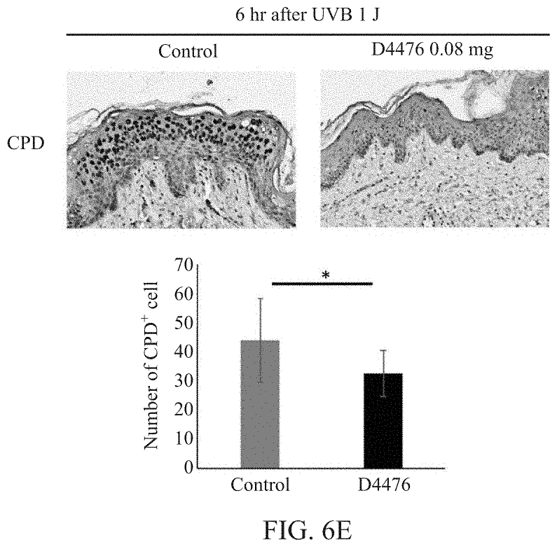

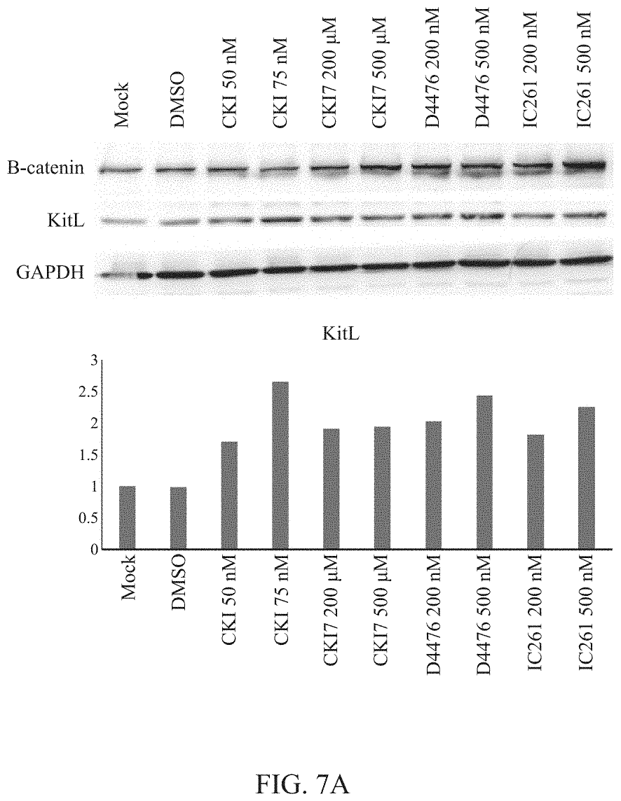

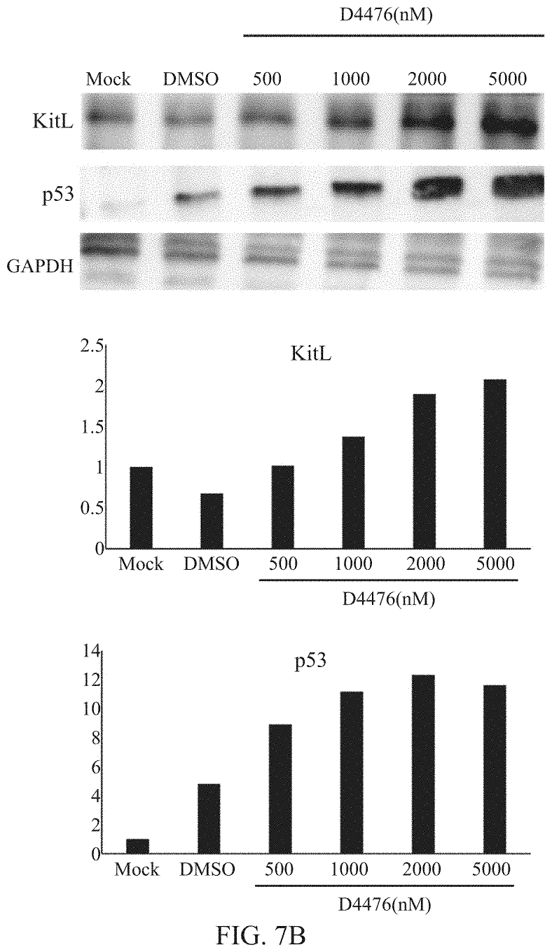

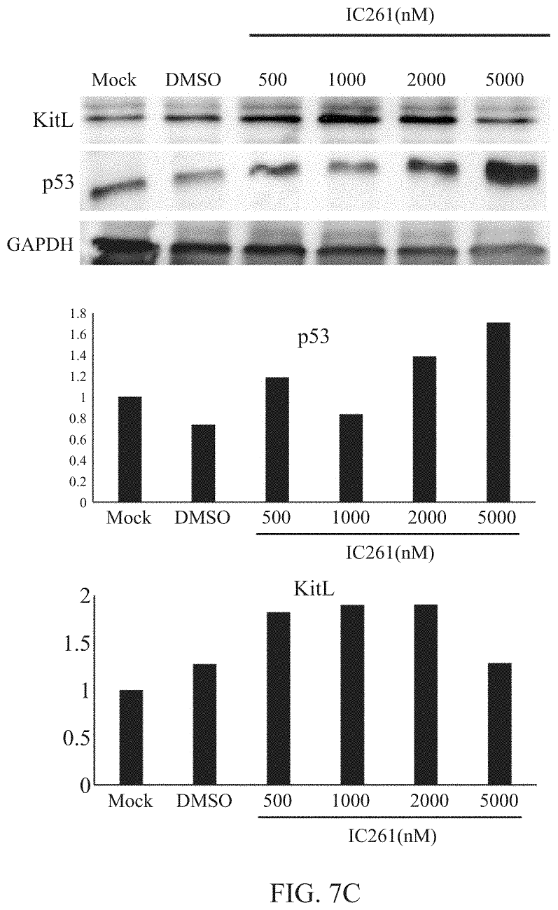

The numbers of Melan-A+cells and Trip-1+cells and also the relative melanin intensity in the control or CKI treatment groups are quantified in the histograms. FIG. 3D shows the Western blotting results and the corresponding quantitated histograms of the total proteins extracted from the epidermis for examining the expression levels of p53 and pigmentation-related proteins. FIG. 3E shows the CPD expression and the quantification of CPD+cell numbers by ImageJ software in UVB-irradiated samples with or without CKI treatment. The statistical results were determined by unpaired t-test analysis and showed as mean±SD. The asterisks *** indicated a significant difference with P<0.001.

FIGS. 4A to 4G show the effect of CKI treatment on human primary keratinocytes and melanocytes. FIG. 4A shows the experimental design of the CKI treatment. In FIG. 4B, the primary human keratinocytes were treated with indicated concentrations of CKI when the cell confluence was 90%. The cell lysates were analyzed by Western blotting, demonstrating p53 activation and increased KitL and c-Kit expressions in a dose-dependent manner. In FIG. 4C, the culture medium was collected after 72 hours of treatment and subjected to ELISA assay, and concentrations of KitL were shown to increase by CKI treatment. Statistical results from a total of 3 independent experiments were determined with unpaired t-test analysis and shown as mean±SD. “NS” indicates no significant difference and asterisks *** indicates a significant difference with P<0.001. In FIGS. 4D and 4E, the primary melanocytes were cultured in conditional medium that have cultured keratinocytes for 3 days and treated with or without CKI.

FIG. 4D shows the results of melanosomes stained with anti-HMB45 antibody; cell numbers were counted by trypan blue stain, and dendrite length was measured on HMB45-stained cells. In FIG. 4E, melanocytes cultured with conditional medium derived from keratinocytes treated with various CKI concentrations show increased expression levels of melanocyte specific regulators, tyrosinase and MITF. In FIG. 4F, the primary human melanocytes were treated with different concentrations of CKI when the cell confluency was 90%. The total proteins were extracted after 72 hours of treatment and examined for expressions of the melanocyte maturation makers including c-Kit receptor, MITF, tyrosinase and phosphorylated MKK6. The Western blotting results showed increased expression of MITF, tyrosinase and p-MKK6 in a dose-dependent manner. In FIG. 4G, the primary melanocytes were subjected to the migration assay. After melanocyte attachment, the medium was changed to those with solvent control or 50 nM CKI. The inserts were removed after 24 hours, and photos were taken at 0, 24, and 48 hours. The data showed that CKI enhanced melanocyte migration.

FIGS. 5A and 5B show the effect of D4476 and IC261 treatments on MC1R mutant mice. FIG. 5A illustrates the experimental design, and FIG. 5B shows the Western blotting analysis of expression levels of proteins in the KitL/c-Kit pathway and melanocyte-related targets.

FIGS. 6A to 6E show the UV protection effect of topical treatment of another CKI, D4476, on human skin explants. FIG. 6A shows the experimental design of D4476 topical application scheme, including 0.04 mg twice for a total of 0.08 mg or 0.05 mg twice for a total of 0.1 mg on three explants, respectively. FIG. 6B shows the histological analysis by Fontana-Masson staining result of D4476 treated skin. FIGS. 6C and 6D show the Western blotting analysis results and the quantitated amount, respectively, of the expression levels of proteins in the c-Kit pathway and melanogenesis-related protein. FIG. 6E shows the CPD staining results of the skin specimen receiving topical D4476 for a total of 0.08 mg, and the histograms show the quantitated number of cells stained with CPD (number of CPD+cell).

FIGS. 7A to 7C show the effects of additional casein kinase 1 inhibitors including A51, CKI7, D4476 and IC261 on the Kit pathway and melanogenesis-related protein expression levels of human keratinocytes.

### DETAILED DESCRIPTIONS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the above examples for carrying out this disclosure without contravening its scope, for different aspects and applications.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

It is further noted that, as used in this disclosure, the singular forms “a,” “an,” and “the” include plural referents unless expressly and unequivocally limited to one referent.

The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

The terms “subject,” “patient” and “individual” are used interchangeably herein and refer to a warm-blooded animal including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In some embodiments, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. In some embodiments, the beneficial effects that a subject derives from a prophylactic or therapeutic agent do not result in a cure of the disorder, disease, or condition.

The term “contacting” or “contact” is meant to refer to bringing together of a cosmetic or therapeutic agent and cell or tissue such that a physiological and/or chemical effect takes place as a result of such contact. Contacting can take place in vitro, ex vivo, or in vivo. In some embodiments, a cosmetic or therapeutic agent is in contact with a cell in a cell culture (in vitro) to determine the effect of the cosmetic or therapeutic agent on the cell. In some embodiments, the contacting of a cosmetic or therapeutic agent with a cell or tissue includes the administration of a cosmetic or therapeutic agent to a subject having the cell or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit abiological or medical response of abiological molecule (e.g., aprotein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier” or “physiologically acceptable excipient” refers to a cosmetically or pharmaceutically acceptable material, composition, or vehicle, such as a liquid or solid filler, diluent, solvent, or encapsulating material. In some embodiments, each component is “pharmaceutically acceptable” in the sense of being compatible with the other ingredients of a cosmetic or pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, Remington: The Science and Practice of Pharmacy, 22nd ed.; Allen Ed.: Philadelphia, Pa., 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more cosmetically or pharmaceutically acceptable excipients, to a subject for preventing, ameliorating or treating one or more symptoms of a disorder, disease, or condition. As used herein, “active ingredient” and “active substance” may be an optically active isomer of a compound described herein.

The terms “drug,” “cosmetic agent” and “therapeutic agent” refer to a compound or a cosmetically or pharmaceutical composition thereof, which is administered to a subject for preventing, ameliorating or treating one or more symptoms of a disorder, disease, or condition.

Casein kinase 1 a (CK1α ), encoded by the Csnklal gene, is a component of the 3-catenin-degradation complex and a regulator of the Wnt signaling pathway. Its ablation induces both Wnt and p53 activation. CK1α phosphorylates $-catenin at Ser45, which primes it for subsequent phosphorylation by GSK-313. GSK-313 destabilizes $-catenin by phosphorylating it at Ser33, Ser37, and Thr41, marking $-catenin for ubiquitination by SCF$-TrCP E3 and proteasomal degradation. This CK1α -dependent phosphorylation functions as a molecular switch for the Wnt pathway. A homozygous deficiency of CK1α results in embryonic lethality, suggesting a fundamental role for CK1α in embryogenesis. In a study of murine intestine epithelium, a CK1α deficiency was found to induce Wnt activation, cell senescence and DNA damage response, with robust p53 activation and cellular senescence in many types of tissues including tissue stem cells. These facts suggest that CK1α involves in cellular processes in various tissues, which is, at least, partly coordinated with p53.

The well-known tumor suppressor protein, p53, is a transcription factor that involves in cellular responses to genotoxic stress and DNA damage. In the skin, p53 also acts against UV damage. UV radiation leads to activation of p53, and p53 stimulates transcriptional upregulation of the proopiomelanocortin (POMC) gene, which is post-translationally processed to adrenocorticotrophic hormone (ACTH), a-melanocyte-stimulating hormone (a-MSH), and P-endorphin. Secreted a-MSH binds to the melanocortin 1 receptor (MCIR) on melanocytes, leading to production of melanin.

The melanin is packaged within melanosomes and transported back to keratinocytes, where they localize over the nucleus as part of the protective tanning response to UV radiation. Secreted u-melanocyte-stimulating hormone (u-MSH) from keratinocytes binds melanocortin 1 receptor (MC1R) on melanocytes, leading to upregulation of cAMP, which stimulates expression of microphthalmia-associated transcription factor (MITF). MITF then transcriptionally activates expression of enzymatic machinery including tyrosinase and tyrosinase-related protein 1 (Tyrp1). Therefore, in the skin, p53 also acts against UV damage via the p53/POMC/a-MSH/MC1R/MITF skin tanning pathway and through the DNA repair/cell cycle arrest/apoptotic pathway.

UV irradiation is recognized as a major extrinsic factor for skin cancers. The melanin in the skin may involve in protection from photoaging and photocarcinogenesis.

Eumelanin is the natural sunscreen induced during sun exposure. Human melanocytes synthesize eumelanin, the dark brown form of melanin, as well as pheomelanin, which is reddish-yellow in color. The relative rates of eumelanin and pheomelanin synthesis by melanocytes determine skin color and the sensitivity of skin to the drastic effects of solar UV. Eumelanin is more stable and less prone to photodegradation than pheomelanin. Eumelanin, but not pheomelanin, is highly efficient as a scavenger of ROS. Pheomelanin pigment pathway produces ultraviolet-radiation-independent carcinogenic contributions to melanomagenesis by a mechanism of oxidative damage.

There is inverse relationship between eumelanin content and the generation of cyclobutane pyrimidine dimers (CPD), the major form of DNA photoproducts.

The MC1R, its agonists, and the signaling pathway involved play a role in regulating the synthesis of eumelanin. Loss-of-signaling MC1R polymorphisms are commonly found among fair-skinned, sun-sensitive and skin cancer-prone populations (e.g., Northern Europeans), who can synthesize pheomelanin but very rare eumelanin.

The most prevalent MC1R mutations (D84E, R151C, R160W and D294H) are commonly referred to as “RHC” (red hair color) alleles because of their association with red hair color, freckling and tendency to burn after UV exposure. Loss-of-signaling MC1R alleles such as the RHC variants are associated with up to a four-fold increased lifetime risk of melanoma and other skin cancers.

As disclosed herein, removal of CK1α , a component of the P-catenin degradation complex, resulted in stabilization of p53, and induced p53-dependent KitL expression in keratinocytes. KitL is a paracrine factor working on receptor c-kit on melanocytes to initiate melanogenesis process and to increase the eumelanin production and skin hyperpigmentation. The eumelanin induced by CK1u inhibition efficiently protects skin from acute sunburn with decreased apoptosis in keratinocytes and reduced pro-inflammatory cytokine production in the mouse model. Inhibition of CK1α in keratinocytes activates a different pathway, p53/KitL/Kit, and induces production of protective eumelanin without the procarcinogenic pheomelanin. Inhibition of CK1α is therefore expected to be an UV-sparing strategy for skin protection from sunlight and for rescuing eumelanin formation in the population with impaired MC1R or with impaired signaling pathway involving MC1R. Inhibition of CK1α not only demonstrates the potential to increase eumelanin, but also shows increase in melanocyte cell number and provides an approach for treating depigmenting diseases, such as vitiligo.

In at least one embodiment, provided herein is a method for increasing a eumelanin level in a subject, comprising administering to the subject a therapeutically effective amount of a casein kinase 1 inhibitor (CKI). In some embodiments, CKI is topically administered to the subject. In some embodiments, CKI is topically administered to a skin of the subject. In some embodiments, the method provided herein for increasing the eumelanin level is to increase the eumelanin level selectively over the pheomelanin level. In some embodiments, the method provided herein for increasing the eumelanin level is to increase the eumelanin level at least ten or more times more than the pheomelanin level. In some embodiments, CKI is topically administered to a selected area of a skin of a subject to increase the eumelanin level at the targeted body parts of the subject.

In at least one embodiment, provided herein is a method for preventing ameliorating, or treating a skin disorder, disease, or condition in a subject, comprising administering to the subject a cosmetically or therapeutically effective amount of a casein kinase 1 inhibitor (CKI). In some embodiments, the administration of the cosmetically or therapeutically effective amount of CKI is topically administered to the subject.

In at least one embodiment, the skin disorder, disease, or condition is caused by UV overexposure. In some embodiments, the skin disorder, disease, or condition is solar erythema, solar allergy, solar urticaria, solar elastosis, photoaging, or a sunburn.

In some embodiments, the skin disorder, disease, or condition is a sunburn. In some embodiments, the skin disorder, disease, or condition is an acute sunburn. In some embodiments, the skin disorder, disease, or condition is hypopigmentation, post-inflammatory hypopigmentation or post-wounding hyperpigmentation. In some embodiments, the skin disorder, disease, or condition is hypomelanosis, idiopathic guttate hypomelanosis, piebaldism, pityriasis alba, pityriasis versicolor, progressive macular hypomelanosis, vitiligo, or Waardenburg syndrome. In some embodiments, the skin disorder, disease, or condition is vitiligo. In some embodiments, the skin disorder, disease, or condition is a skin cancer. In some embodiments, the skin disorder, disease, or condition is actinic keratosis, atypical mole, basal cell carcinoma (BCC), melanoma, Merkel cell carcinoma (MCC), squamous cell carcinoma (SCC), or cutaneous malignant melanoma.

In some embodiments of the present disclosure, the methods provided herein comprise treating a subject regardless of patient's age, although some diseases or disorders are more common in certain age groups.

In some embodiments of the present disclosure, provided herein is a method for protecting a subject from ultraviolet radiation, comprising administering to the subject an effective amount of a casein kinase 1 inhibitor. In some embodiments, provided herein is a method for increasing skin pigmentation in a subject, comprising administering to the subject a cosmetically or therapeutically effective amount of a casein kinase 1 inhibitor. In some embodiments, the methods for protecting a subject from ultraviolet radiation and for increasing skin pigmentation comprise administration of a cosmetically or therapeutically effective amount of CKI that is topically administered to the subject. In some embodiments, CKI is topically administered to a selected area of a skin of a subject to effect at the targeted body parts of the subject.

The methods of preventing or treating in the present disclosure are effected by contacting/administering an agent capable of inhibiting CK1. CK1 is a well-conserved family of Ser/Thr kinases found in every organism tested, from yeast to man. In mammals, the CK1 family is composed of seven genes (u, R, y1, y2, y³, 6, and F) encoding 11 alternatively spliced isoforms. Members of the CK1 family share a conserved catalytic domain and ATP-binding site, which exclusively differentiate them from other kinase families. CK1 is a ubiquitous enzyme found in all cells, occupies different sub-cellular localizations and is involved in various cellular processes besides Wnt signaling.

In some embodiments, the CK1 inhibitors (CKI) increase the expression and/or activity of p53 (by at least 2 folds) and/or activate a DNA damage response (DDR). CK1 inhibitors (CKIs) of the present disclosure may have at least twice, at least 5 times, or at least 10 times the inhibitor activity towards CK1 as compared to other kinases such as cyclin-dependent kinases (CDKs) regulating the cell cycle, (e.g., Cdk2, Cdk4, and Cdk6). In addition, CK1 inhibitors have at least twice, at least 5 times, or at least 10 times the inhibitor activity towards CK1 as compared to protein kinase C (PKC), PKA, HER2, raf-1, MEK1, MAP kinase, EGF receptor, PDGF receptor, IGF receptor, PI3 kinase, Weel kinase, Src, and/or Abl.

CKI are selective towards CK1-u (CSNK1A; at the genomic, mRNA or protein level, GenBank Accession Nos. NP_001020276, NM_001025105 and NM_001020276). Thus, for example, such CK1 inhibitors have at least twice, at least 5 times, or at least 10 times the inhibitor activity towards CK1-u as compared to CK1-6 and CK1-8.

In at least one embodiment, a casein kinase 1 inhibitor has the general formula I, including any stereoisomer or salt thereof:

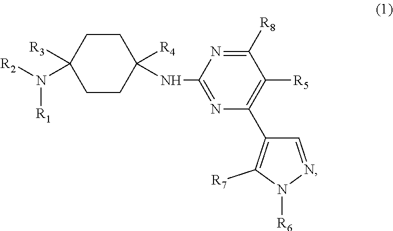

wherein: R₁ and R₂ are each independently selected from the group consisting of H, straight or branched C1-C8 alkyl, straight or branched C1-C5 alkoxy, straight or branched C1-C5 acyl, C5-C15 aryl, and C3-C7 heteroaryl each optionally substituted by at least one 20 of halide, hydroxyl, ester, ether, C5-C15 aryl, C3-C7 heteroaryl, and amide; or R₁ and R₂ together with the nitrogen atom they are connected to form a 4-7 membered saturated, unsaturated or aromatic ring that may optionally include at least one of N, 0, NH, C=N, C═O and SO2 and can optionally be substituted with at least one of straight or branched C1-C5 alkyl, C5-C15 aryl, C3-C7 heteroaryl, hydroxyl, halide and cyano; R3 and R₄ are each independently selected from the group consisting of H, straight or branched C1-C8 alkyl optionally substituted by at least one of halide, hydroxyl, alkoxy, C5-C15 aryl, C3-C7 heteroaryl, ester and amide; or R₁ or R₂ together with R₃ and the carbon and nitrogen atom they are each connected to form a 4-7 membered saturated, unsaturated or aromatic ring that may optionally include at least one of N, NH, 0, C=N, C=0, and S02, and can optionally be substituted with at least one of straight or branched C1-C5 alkyl, C5-C15 aryl, C3-C7 heteroaryl, hydroxyl, carbonyl, and halide; Rₛ and Rs are each independently selected from the group consisting of H, halide, straight or branched C1-C8 alkyl, straight or branched C2-C8 alkenyl, and straight or branched C2-C8 alkynyl optionally substituted by at least one halide; R₆ is selected from straight or branched C1-C8 alkyl, straight or branched C2-C8 alkenyl, straight or branched C2-C8 alkynyl, C5-C10 cycloalkyl, and saturated or unsaturated 4-6 membered heterocycle optionally substituted by at least one of straight or branched C1-C8 alkyl, C3-C7 cycloalkyl, 4-6 membered heterocycle, C5-C15 aryl, C3-C7 heteroaryl, halide, hydroxyl, and C1-C5 alkyl halide; R₇ is selected from the group consisting of straight or branched C1-C8 alkyl, straight or branched C2-C8 alkenyl, and straight or branched C2-C8 alkynyl optionally substituted by at least one of C3-C7 cycloalkyl, 4-6 membered heterocycle, C5-C15 aryl, C3-C7 heteroaryl, halide, hydroxyl, and C1-C5 alkyl halide.

Additional casein kinase I inhibitors include those described in International Publication No. WO 2017/021969; the disclosure of which is incorporated herein by reference in its entirety.

In some embodiment, the casein kinase 1 inhibitor is selected from the group consisting of CKI7, D4476, IC261, and a compound represented by formulas I to VII:

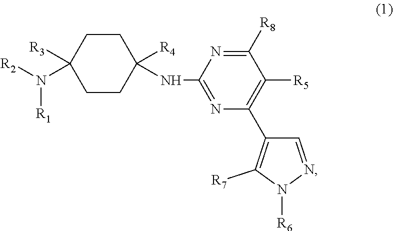

wherein: R₁ and R₂ are each independently selected from the group consisting of H, straight or branched C1-C8 alkyl, straight or branched C1-C5 alkoxy, straight or branched C1-C5 acyl, C5-C15 aryl, and C3-C7 heteroaryl each optionally substituted by at least one of halide, hydroxyl, ester, ether, C5-C15 aryl, C3-C7 heteroaryl, and amide; or R₁ and R₂ together with the nitrogen atom they are connected to form a 4-7 membered saturated, unsaturated or aromatic ring optionally including at least one of N, 0, NH, C=N, C═O and SO₂ and optionally substituted with at least one of straight or branched C1-C5 alkyl, C5-C15 aryl, C3-C7 heteroaryl, hydroxyl, halide and cyano; R₃ and R₄ are each independently selected from the group consisting of H, straight or branched C1-C8 alkyl optionally substituted by at least one of halide, hydroxyl, alkoxy, C5-C15 aryl, C3-C7 heteroaryl, ester and amide; or R₁ or R₂ together with R₃ and the carbon and nitrogen atom they are each connected to form a 4-7 membered saturated, unsaturated or aromatic ring optionally including at least one of N, NH, 0, C=N, C=0, and SO₂, and optionally substituted with at least one of straight or branched C1-C5 alkyl, C5-C15 aryl, C3-C7 heteroaryl, hydroxyl, carbonyl, and halide; Rₛ and Rs are each independently selected from the group consisting of H, halide, straight or branched C1-C8 alkyl, straight or branched C2-C8 alkenyl, and straight or branched C2-C8 alkynyl optionally substituted by at least one halide; R₆ is selected from the group consisting of straight or branched C1-C8 alkyl, straight or branched C2-C8 alkenyl, straight or branched C2-C8 alkynyl, C5-C10 cycloalkyl, and saturated or unsaturated 4-6 membered heterocycle optionally substituted by at least one of straight or branched C1-C8 alkyl, C3-C7 cycloalkyl, 4-6 membered heterocycle, C5-C15 aryl, C3-C7 heteroaryl, halide, hydroxyl, and C1-C5 alkyl halide; R₇ is selected from the group consisting of straight or branched C1-C8 alkyl, straight or branched C2-C8 alkenyl, and straight or branched C2-C8 alkynyl optionally substituted by at least one of C3-C7 cycloalkyl, 4-6 membered heterocycle, C5-C15 aryl, C3-C7 heteroaryl, halide, hydroxyl, and C1-C5 alkyl halide;

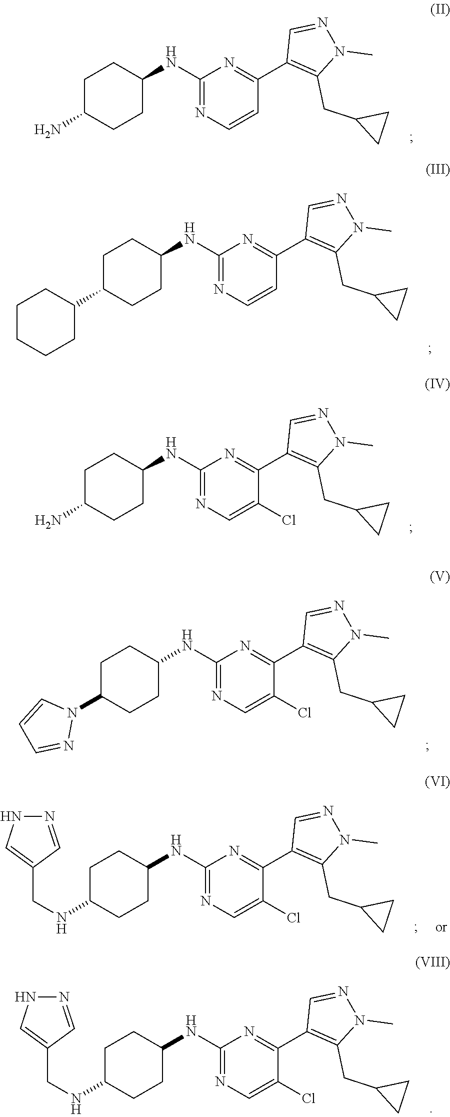

The topical administration, as used herein, includes (intra)dermal, conjunctival, intracorneal, intraocular, ophthalmic, auricular, transdermal, nasal, vaginal, urethral, respiratory, and rectal administration.

The cosmetically or pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches.

The topical formulation of the cosmetically or pharmaceutical compositions provided herein can also comprise liposomes, micelles, microspheres, nanosystems, and any mixtures thereof.

Cosmetically or pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The cosmetic or pharmaceutical compositions can also be administered topically by electroporation, iontophoresis, phonophoresis, sonophoresis, microneedle or needle-free injection, such as PowderTect (Chiron Corp., Emeryville, Calif.) and Bioject (Bioject Medical Technologies Inc., Tualatin, OR).

The cosmetic or pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase.

The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier.

Suitable gelling agents include, but are not limited to, crosslinked acrylic acid polymers, such as carbomers, carboxypolyalkylenes, and Carbopol; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

### EXAMPLE

Exemplary embodiments of the present disclosure are further described in the following examples, which should not be construed to limit the scope of the present disclosure.

Generally, the nomenclature used herein and the laboratory procedures utilized in the present disclosure include molecular, biochemical and recombinant DNA techniques. Such techniques are thoroughly explained in the literature. See, for example, “Molecular Cloning: A laboratory Manual,” Sambrook et al., (1989); “Current Protocols in Molecular Biology,” Volumes I-III Ausubel, R. M., ed. (1994); “A Practical Guide to Molecular Cloning,” John Wiley & Sons, New York (1988); Watson et al., “Recombinant DNA,” Scientific American Books, New York; Birren et al. (eds) “Cell Biology: A Laboratory Handbook,” Volumes I-III Cellis, J. E., ed. (1994); “Culture of Animal Cells - A Manual of Basic Technique” by Freshney, Wiley-Liss, N.Y. (1994), Third Edition; “Transcription and Translation,” Hames, B. D., and Higgins S. J., eds. (1984); “Animal Cell Culture,” Freshney, R. I., ed. (1986); “Immobilized Cells and Enzymes,” IRL Press, (1986); “A Practical Guide to Molecular Cloning,” Perbal, B., (1984) and “Methods in Enzymology,” Vol. 1-317, Academic Press; “PCR Protocols: A Guide To Methods and Applications,” Academic Press, San Diego, Calif. (1990); Marshak et al., “Strategies for Protein Purification and Characterization - A Laboratory Course Manual,” CSHL Press (1996); all of which are incorporated by reference as if fully set forth herein. Other general references are provided throughout this disclosure. The procedures therein are believed to be well known in the art and are provided for the convenience of the reader. All the information contained therein is incorporated herein by reference.

Materials and Methods Mice The Tyr-CreER/FusionRed mice are designed for tracing melanocyte stem cells by FusionRed fluorescence from Cre-recombination driven by melanocyte-specific tyrosinase promoter. C57BL/6J-Mc1r^{em1} mice, which have the mutated Mclr gene with a deletion of one single nucleotide at position 549 leading to 12 amino acids out of frame mutation, were generated using CRISPR-Cas9 system. Then, the MC1R mutant mouse with yellow coat color was crossed with K14-CreER;CK1a^{f}/f mouse to generate K14-CreER;CK1a^{ff};MC1RKxIvK mouse. The mutant mice and the wild-type C57BL/6J mice were generated in National Laboratory Animal Center in Tainan, Taiwan.

Experiments and animal caring were performed in Laboratory Animal Center at Tzu Chi University, Hualien, Taiwan.

CKI and its preparation for topical use The small molecule inhibitor targeting CKla (CKI) used in this disclosure was developed and gifted by Dr. Yinon Ben-Nerian. The details of CKI were published (Minzel W., Venkatachalam A., Fink A., et al. “Small Molecules Co-targeting CK1α and the Transcriptional Kinases CDK7/9 Control AML in Preclinical Models.” Cell. 2018; 175(1):171-185).

For topical application on mouse skin in this disclosure, 1 mg of CKI was dissolved in 6 μL of dimethyl sulfoxide (DMSO) and mixed with 54 μL of vehicle that contains 60% of white wax and 40% of paraffin oil.

For topical use on human skin explant, a CKI solution containing 1 mg in 6 μL DMSO was diluted with 594 μL phosphate buffered saline (PBS) for further use. The final working concentration is 0.01 mg/6 μL.

In addition, other compounds with known casein kinase 1 inhibition activity were also tested in this disclosure, including D4476 and IC261. D4476 was first reported by Rena G., et al. in the journal article “D4476, a cell-permeant inhibitor of CK1, suppresses the site-specific phosphorylation and nuclear exclusion of FOXOla.” EMBO Rep. 2004 January; 5(1):60-5, while IC261 was reported by. Mashhoon N., et al. in the journal article “Crystal structure of a conformation-selective casein kinase-1 inhibitor.” J. Biol. Chem. 2000 Jun 30; 275(26):20052-60, and both molecules can be obtained commercially.

Human skin explant The human foreskins were used for in vitro explant culture experiments. IRB was approved by Research Ethics Committee, Hualien Tzuchi Hospital (IRB107-51-A).

After removing the fat, the specimens were transferred to the plates coated with 0.1% gelatin in advance. Foreskin explants were cultured with Dulbecco's Modification of Eagle's Medium (DMEM) supplemented with 20% fetal bovine serum (FBS) in 37° C., in humidified incubators supplemented with 5% CO2. After 24 hours, 0.01 mg of CKI was dropped onto the cultured skin for two times at one day apart, with a total of 0.02 mg CKI applied. Five days after CKI administration, the tissue was fixed with 4% paraformaldehyde (PFA), and then embedded with paraffin for histology examination.

In addition, the epidermis and dermis were separated by thermolysin. The total proteins of epidermis were extracted by T-PER Tissue Protein Extraction Reagent (78510, Thermo Fisher) for Western blotting analysis.

Fontana-Masson and hematoxylin and eosin staining Skin biopsies were fixed with 4% formalin or paraformaldehyde and embedded in paraffin. The section was trimmed to 5- m thickness by microtome.

Fontana-Masson and hematoxylin and eosin staining were performed by using the kit from ScyTek Laboratories, Inc. according to the manufacturer's instructions.

For Fontana-Masson staining, briefly, the ammoniacal silver solution was pre-warmed in 58 to 60° C. water bath. The sections were deparaffinized and hydrated with distilled water, incubated in warmed ammoniacal silver solution for 30 minutes until yellow color appeared. Then, the slides were incubated in gold chloride solution (0.2%) for 30 seconds, and the melanin stain could then be clearly observed as black color in this step. Finally, the slides were treated with nuclear fast red solution for 5 minutes and then dehydrated with absolute alcohol.

Immunohistochemistry Immunohistochemistry was demonstrated with antigen retrieval solution (citrate pH 6.0, Dako 5236984) in 95° C. for 30 minutes, 3% hydrogen peroxide in ddH₂0 was used for blocking endogenous peroxidase activity. Primary antibody was applied in 4° C. overnight, and then incubated with secondary antibody at room temperature for 30 minutes. The signal was detected by AEC+Substrate-Chromogen (K346111, Dako) or Dako REAL EnVDetectSys Perox/DAB+, Rb/M (K500711, Dako), counterstained with hematoxylin and mounted with either aqueous- or organic-based medium.

For fluorescent staining, after incubation of secondary antibody, a few drops of aqueous mounting medium were wiped with 4′,6-diamidino-2-phenylindole (DAPI) (ChemCruz Biochemicals) and then covered with cover slide to be stored at 4° C. for analysis. Imaging was acquired using fluorescent microscope (Nikon Eclipse Ti2).

Western blot For experiments involving tissues, the tissues were homogenized and lysed in 1x tissue protein extraction reagent (Thermo Fisher) containing protease inhibitor (Millipore) and kept on ice; for experiments involving cells, cell pellet was collected and lysed in 1x radioimmunoprecipitation assay (RIPA) lysis buffer containing protease inhibitor (Millipore) and kept on ice. The total protein concentration was determined using Protein Assay Dye Reagent Concentrate (Bio-Rad) following manufacturer's instruction with a microplate reader. Sample was diluted in SDS-PAGE Sample Buffer (Bio-Rad) and heated at 95° C. for 5 minutes. The protein sample was then resolved on SDS-PAGE gels (TGX FastCast acrylamide solutions, Bio-Rad) and transferred onto poly(vinylidene fluoride) (PVDF) membranes (Millipore). 5% BSA was used, and then the membranes were incubated with primary antibodies overnight at 4° C. The membranes were then washed by Tris-buffered saline with Tween 20 (TBST) and incubated with secondary antibodies for 1 hour at room temperature. The signal was detected using iBright FL1000 Imaging System from Thermo Fisher Scientific.

Antibodies The antibodies used in immunohistochemistry (IHC) and Western blotting (WB) in this disclosure were listed in Table 1 below, where manufacturers, catalogue number and the dilution ratio used were provided.

TABLE 1

| Antibody | Manufacturer | Number | WB | IHC |
|---|---|---|---|---|
| Purified mouse anti-β-catenin | BD, USA | 61054 | 1:1,000 | 1:200 |
| Rabbit anti-c-Kit (CD117) | Invitrogen Thermo Fisher, | 34-8800 | 1:1,000 | 1:200 |
| | USA | | | |
| p53(1C12) mouse mAb | Cell Signaling Technology, | #2524 | 1:1,000 | |
| | USA | | | |
| Glyceraldehyde 3 phosphate | Cell Signaling Technology, | #2118 | 1:2,000 | |
| dehydrogenase (GAPDH) | USA | | | |
| Anti-TRP1 antibody (TA99) | Abcam, UK | ab3312 | 1:1,000 | 1:200 |
| Donkey IgG-heavy and light | Bethyl Laboratories Inc., | A140-107D4 | | 1:500 |
| chain antibody | USA | | | |
| Donkey IgG-heavy and light | Bethyl Laboratories Inc., | A140-107D2 | | 1:500 |
| chain antibody | USA | | | |
| Goat anti-rabbit IgG, | Millipore, Germany | AP132P | 1:5,000 | |
| peroxidase conjugated | | | | |
| Goat anti-mouse IgG, | Millipore, Germany | AP124P | 1:5,000 | |
| peroxidase conjugated | | | | |
| MiTF (SPM290) | Santa Cruz Biotechnology, | sc-56433 | 1:1,000 | 1:50 |
| | Inc., USA | | | |
| Tyrosinase (T311) | Santa Cruz Biotechnology, | sc-20035 | 1:1,000 | |
| | Inc., USA | | | |
| Anti-SCF antibody | Abcam, UK | ab64677 | 1:1,000 | |
| Anti-MKK6 | Cell Signaling Technology, | CST #8550 | 1:1,000 | |
| | USA | | | |
| Anti-pMKK6 | Cell Signaling Technology, | CST #12280 | 1:1,000 | |
| | USA | | | |
| Anti-HMB45 | Ventana | 7904366 | | Ready to use |

UVB irradiation Mice furs were shaved and trimmed before UVB irradiation. 750 mJ/cm² to 1,000 mJ/cm² UVB irradiation was carried out by a microprocessor-controlled UV irradiation system-321 nm (BLX-312 by Witec AG). 24 hours after the irradiation, mouse skin was harvested and analyzed.

Eumelanin and pheomelanin analysis Tail skins from mice completing CKI treatment were obtained for epidermal sheet separation. Skin samples were measured and rinsed with Ca²+-, Mg²+-free PBS (pH 7.4) to remove blood contaminants and were then treated with 0.25% trypsin (Difco; Becton Dickinson Microbiology Systems) in PBS (pH 7.2) for 16 to 18 hours at 28° C. The epidermis and dermis were separated and stored at −80° C. until use.

For assays of melanin content, the tissues were minced with scissors and homogenized in 10 volumes of PBS at 28° C.

Samples of epidermis and dermis were processed for chemical analyses of eumelanin by detecting the specific degradation product, pyrrole-2,3,5-tricarboxylic acid (PTCA), and for chemical analyses of pheomelanin by detecting the specific degradation product, 4-amino-3-hydroxyphenylalanine (4-AHP). One nanogram of PTCA or 4-AHP corresponds to 50 ng of eumelanin or 9 ng of pheomelanin. The statistical significance of differences in the contents of eumelanin and pheomelanin was determined by Student's t test by comparisons of groups of equal size.

Cell culture Human keratinocyte and melanocyte were cultured from human foreskin. The keratinocyte was cultured with keratinocyte serum-free medium (SFM), or K-SFM, which is a complete serum-free medium and supplemented with human recombinant epidermal growth factor (rEGF) and bovine pituitary extract (BPE) (Gibco, 17005042) at the time of use. Melanocyte was cultured with Medium 254 containing human melanocyte growth supplement (HMGS) (Gibco, M254500). The culture was maintained at 37° C. and in 5% CO2. Keratinocyte Kit ligand secretion was detected by Human SCF ELISA Kit (Abcam, ab176109). All of the procedures followed manufacturer's instruction.

Melanocyte migration assay A wound healing assay was adopted to detect cell migration. Specifically, melanocytes were seeded in a culture-insert at a density of 10³ cells per well. After allowing the cells to attach overnight, the culture-insert was removed, and cells were washed with PBS to remove non-adherent cells. Fresh medium without or with CKI was added, followed by incubation for 24 hours. The number of cells that migrated into the wound space was manually counted in three fields per well by a light microscope (Olympus, CKX53). Image J analysis was then used to quantify the areas.

Statistical analysis The statistical analysis in this disclosure present analysis result as mean±standard deviation. Student t test was applied for comparison between groups. P-values less than 0.05 were considered statistically significant.

### Example 1. UV protection effect of topical CKI application on wild-type mice

The C57BL/6 mice were used as experimental model for testing a safety dose of topical CKI. CKI was topically applied on the ears of C57BL/6 mice at an amount of 0.1 mg for each application at a frequency of every other day (Q.O.D.) for 4 weeks.

Therefore, CKI was topically applied 3 times per week, and a total of 0.3 mg CKI was applied per week, with a total of 1.2 mg CKI applied over 4 weeks.

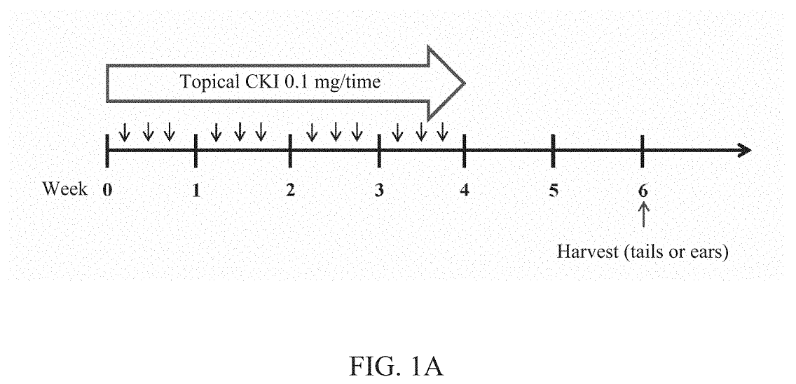

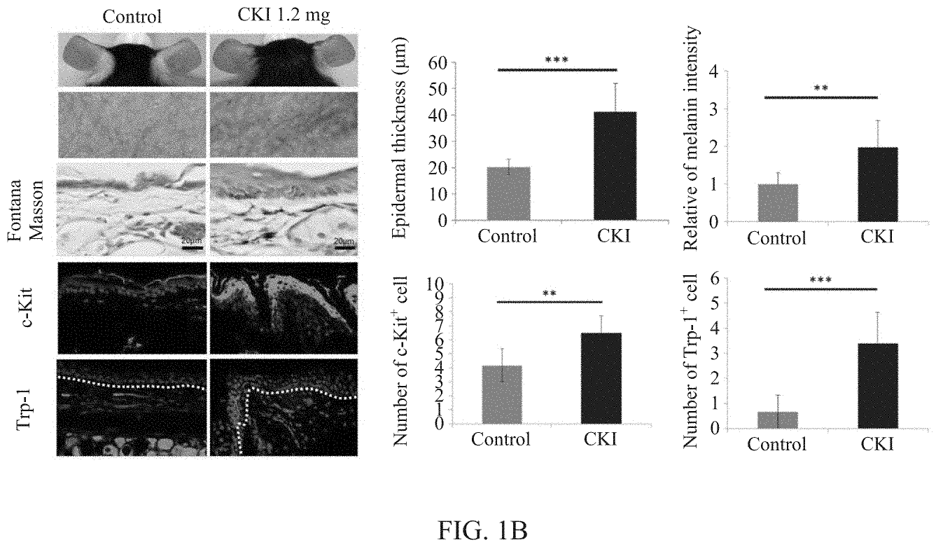

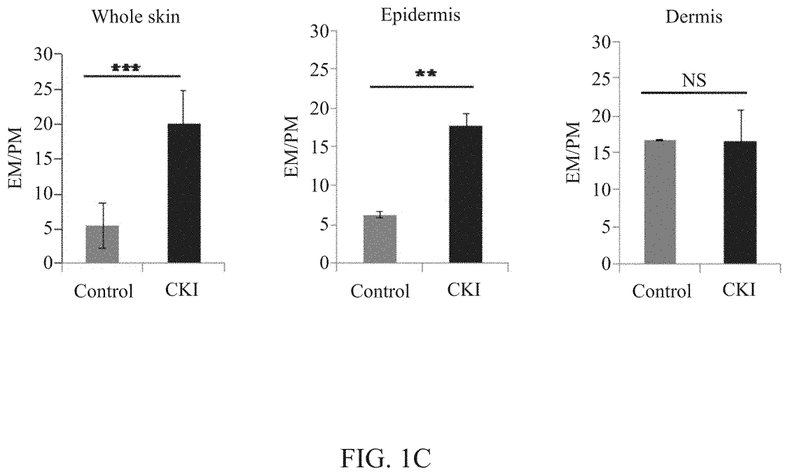

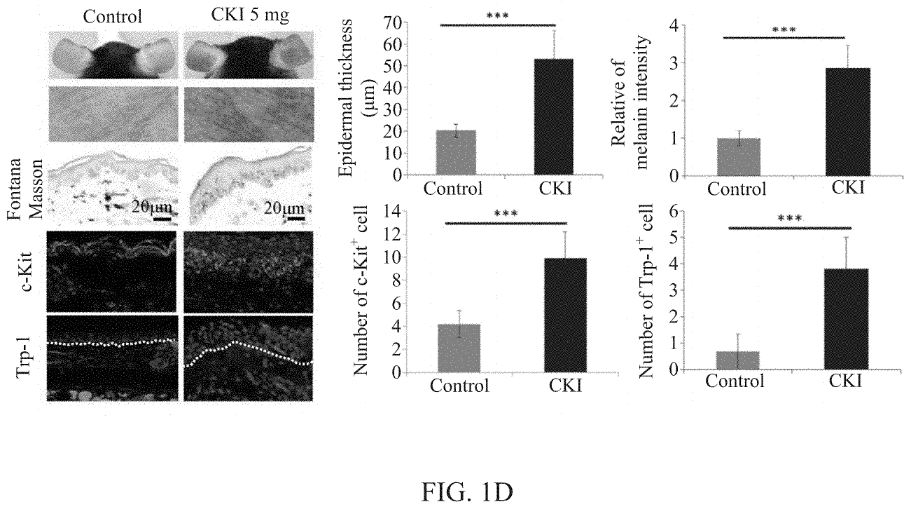

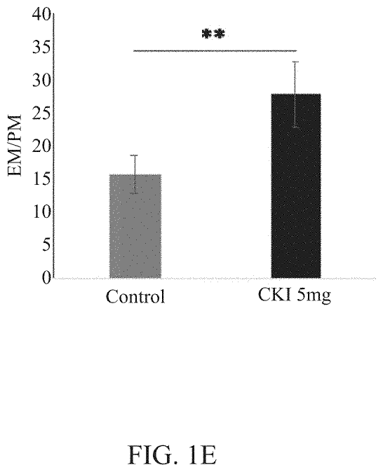

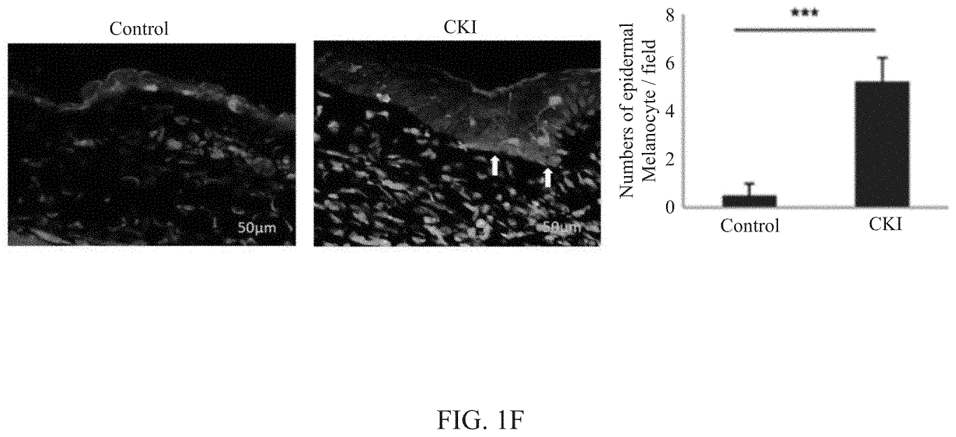

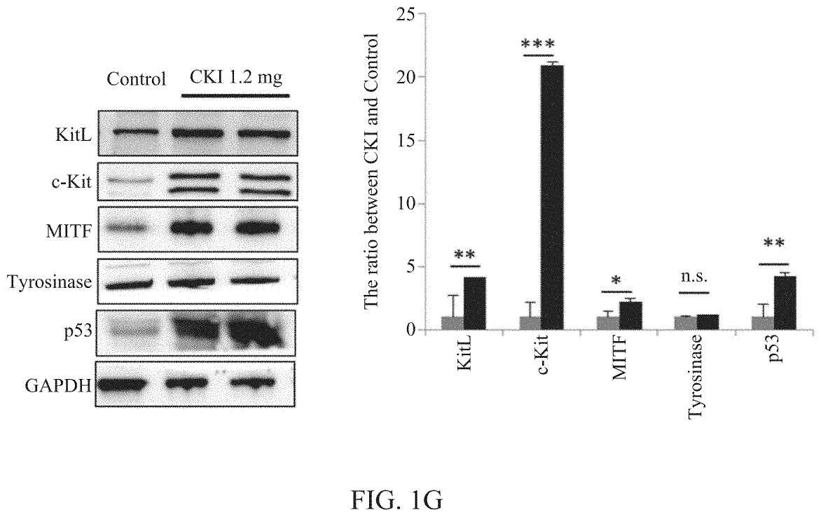

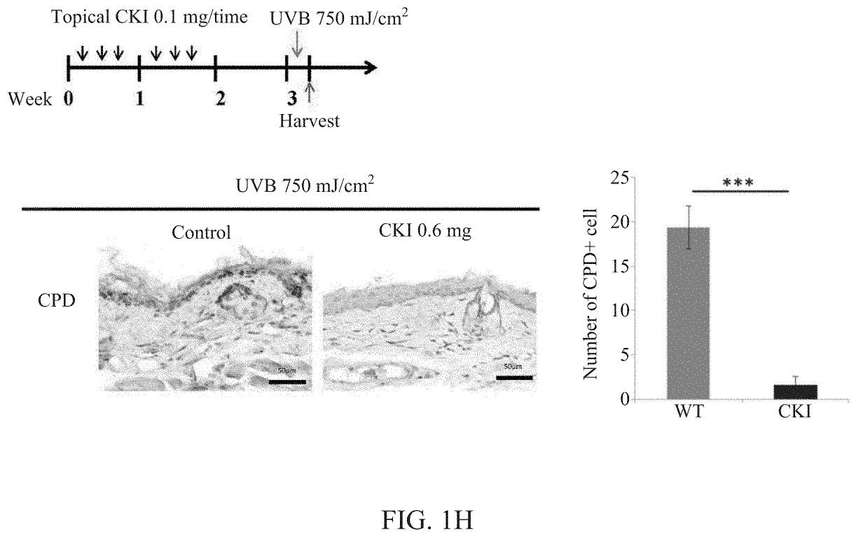

Phenotypes of the mice were recorded every week. Ear samples were harvested for tissue analysis at the end of week 6 after 12 topical CKI applications for a total of 1.2 mg CKI, as shown in FIG. 1A.

As shown in FIG. 1B and FIG. 1D, photographs of the phenotypes showed that skin color of the mice ears became visibly darker after CKI topical treatment compared with the control group; the total dose of CKI treated was 1.2 mg (FIG. 1B) or 5 mg (FIG. 1D). Also shown in FIG. 1B and FIG. 1D, Fontana-Masson staining demonstrated that the amount of melanin in epidermis was already significantly increased after treatment of 1.2 mg CKI and even more intense after treatment of 5 mg CKI.

Furthermore, c-Kit staining showed that receptor KIT in the keratinocytes and melanocytes in the epidermis was also significantly increased. These results revealed that topical CKI increases skin pigmentation and epidermal thickness in the same way as UV tanning.

In mouse skin, melanocytes mostly resided in the dermis. Eumelanin and pheomelanin analysis showed that after topical treatment of 1.2 mg CKI, content of eumelanin was significantly increased in the skin, and the increased eumelanin was mainly localized in the epidermis rather than in the dermis, and there is a significantly increased eumelanin to pheomelanin ratio as shown in FIG. 1C. Therefore, topical CKI application may induce paracrine factors that lead to the migration of melanocyte from dermis into epidermis. A significantly increased eumelanin to pheomelanin ratio was also observed in the mice treated with a total of 5 mg CKI, as shown in FIG. 1E.

To investigate the behavior of melanocyte stem cells, reporter mice Tyr::CreER^{T2}; G/mR were generated. Tamoxifen induction in the reporter mice can cause melanocytes to specifically express red fluorescence on their membrane (Fusion Red), among the other cells in the tissue that express green fluorescence (GFP). Therefore, the reporter mice are used to trace the migration of melanocytes. As shown in FIG. 1F, it was found that before topical CKI treatment, melanocytes were mainly restricted in dermis and rarely in the epidermis (as shown in the left pictures); however, after topical treatment of a total of 1.2 mg CKI, melanocyte numbers in the epidermis significantly increased (as shown in the right graph), indicating that the topical CKI induced the migration of melanocytes from dermis to epidermis. Further, Western blotting analysis of the skin tissue showed that Kit-Ligand (KitL), a factor known for melanocyte migration and survival, was increased after topical CKI treatment, as shown in FIG. 1G. Also, from FIG. 1G, the major melanocyte regulation transcription factor MITF and tyrosinase, the rate-limiting melanogenesis enzyme, were all significantly up-regulated. These results confirmed that topical CKI induced Kit-Ligand production, which promotes melanocyte migration into epidermis and stimulates melanogenesis.

In addition, the photoprotective effects of topical CKI treatment was examined by measuring the amount of cyclobutene pyrimidine dimer (CPD) in skin after UVB exposure. Mouse skin was harvested 24 hours after UVB irradiation of 750 mJ/cm², and the immunohistochemistry analysis of the skin from CKI-treated group with a total of 0.6 mg CKI showed less CPD+cells, which represents damage from UV irradiation, than the control group without CKI treatment, as shown in FIG. 1H. This indicates that topical CKI exerts UV protective function to skin.

### Example 2. CKla inhibition and topical CKI application on MC1R mutant mice

induces melanin production and protects MC1R mutant mice from UVB-induced DNA damage Melanocortin receptors (MCR) belong to the G-protein couple receptor family, and have been classified into five members according to their function and tissue expression, including MC1R, MC2R, MC3R, MC4R and MC5R. In the role of UV protection, MC1R is activated by a peptide hormone called melanocortin derived from proopiomelanocortins (POMCs). u-MSH is one type of melanocortin secreted from keratinocyte, and is responsible for human skin pigmentation. Therefore, the POMC/u-MSH/MC1R pathway is involved in the production of eumelanin. As shown above, CK1α inhibition with topical application of CKI activates KitL/Kit pathway, and it is further investigated to show the effects of CK1α inhibition in the mice carrying MC1R mutation.

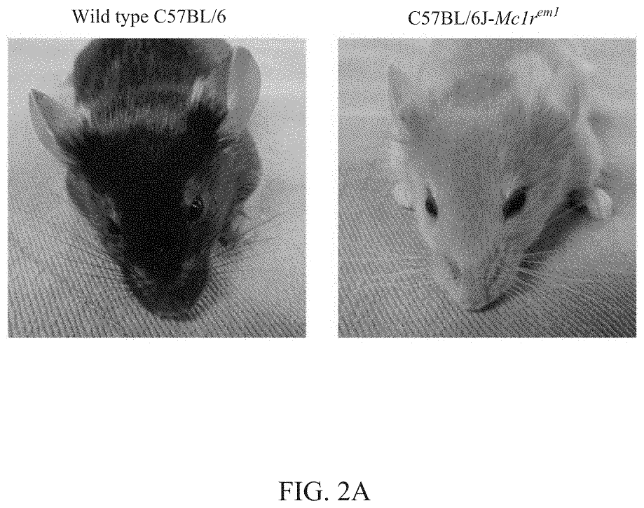

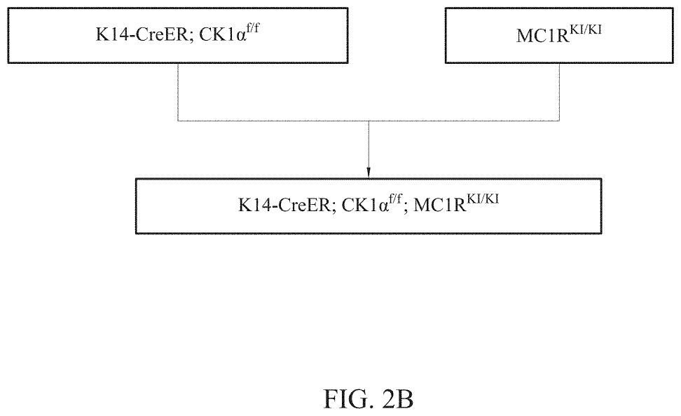

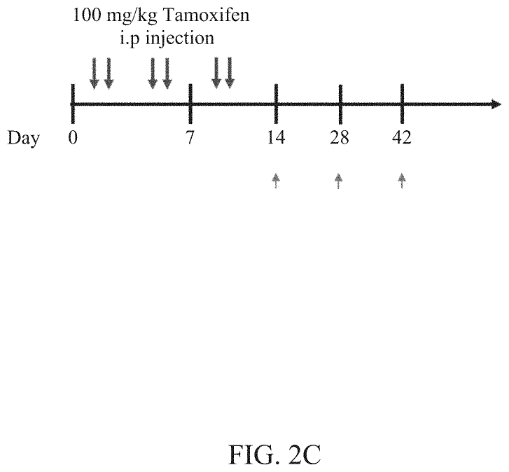

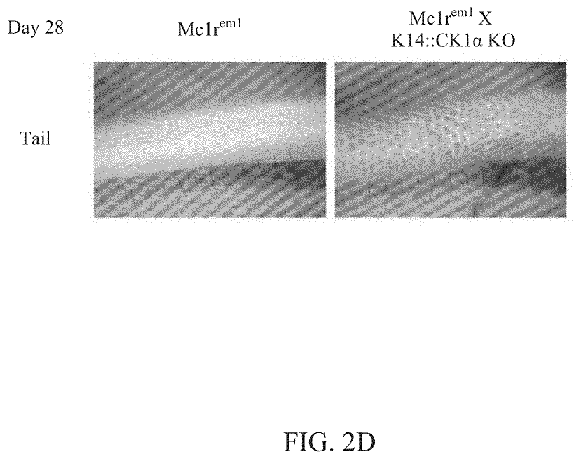

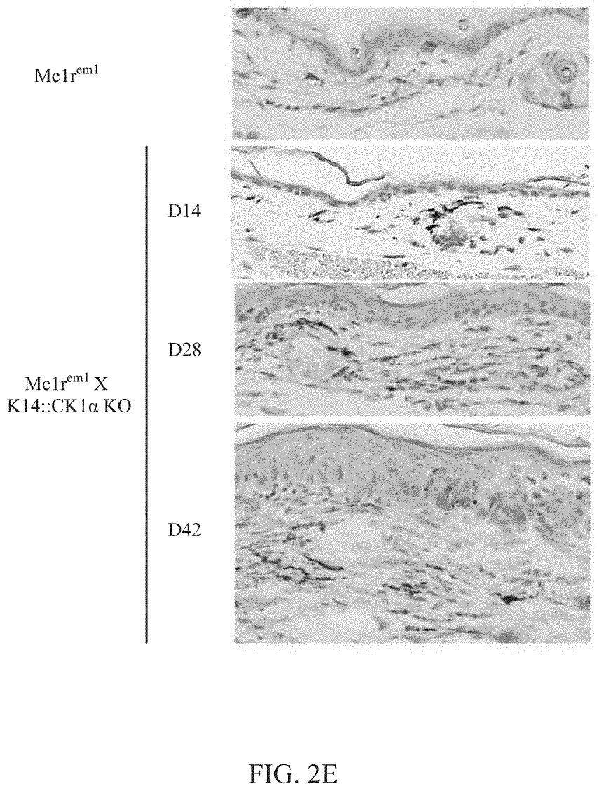

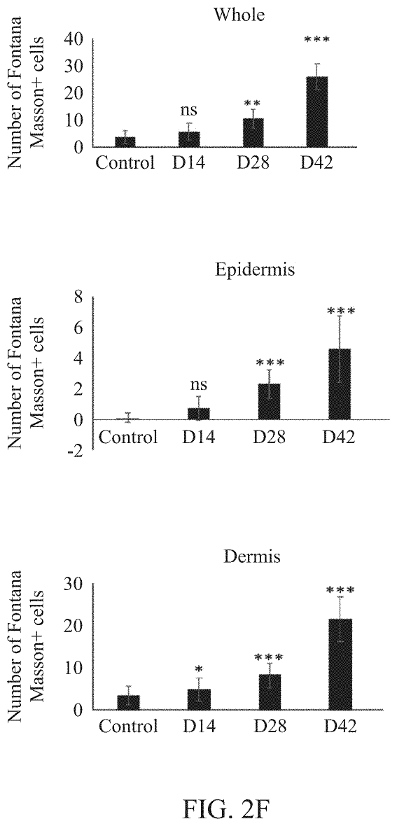

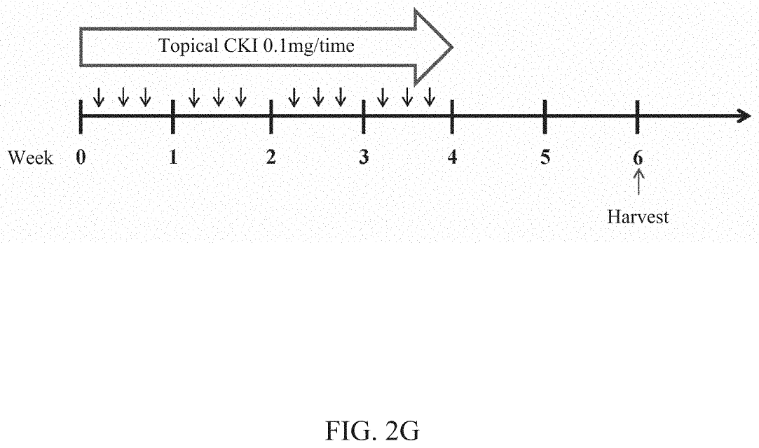

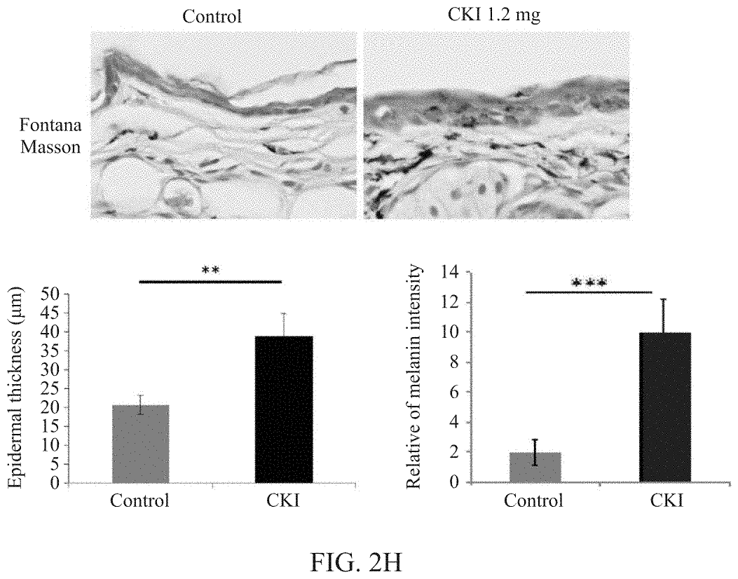

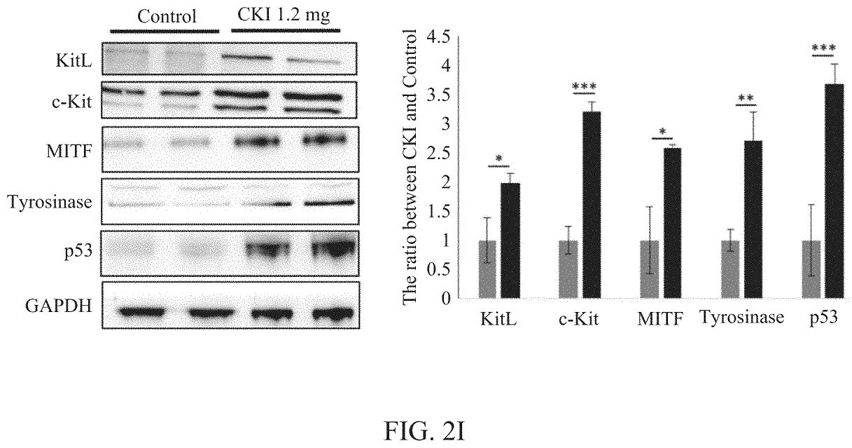

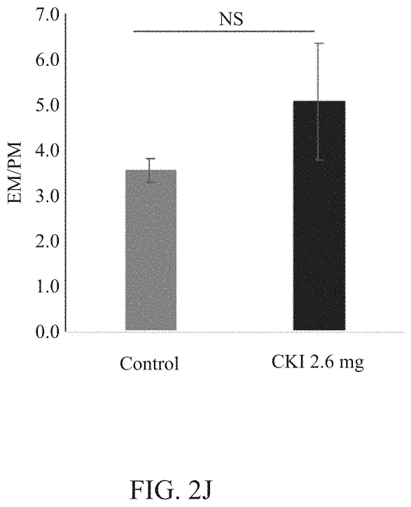

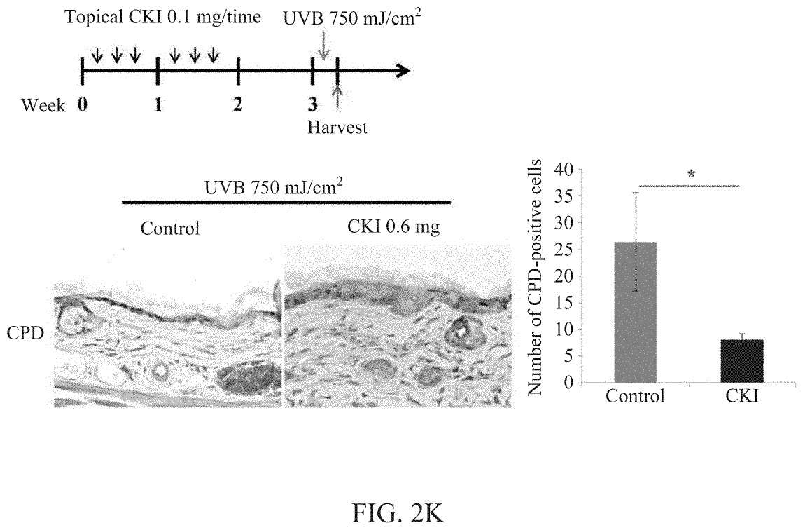

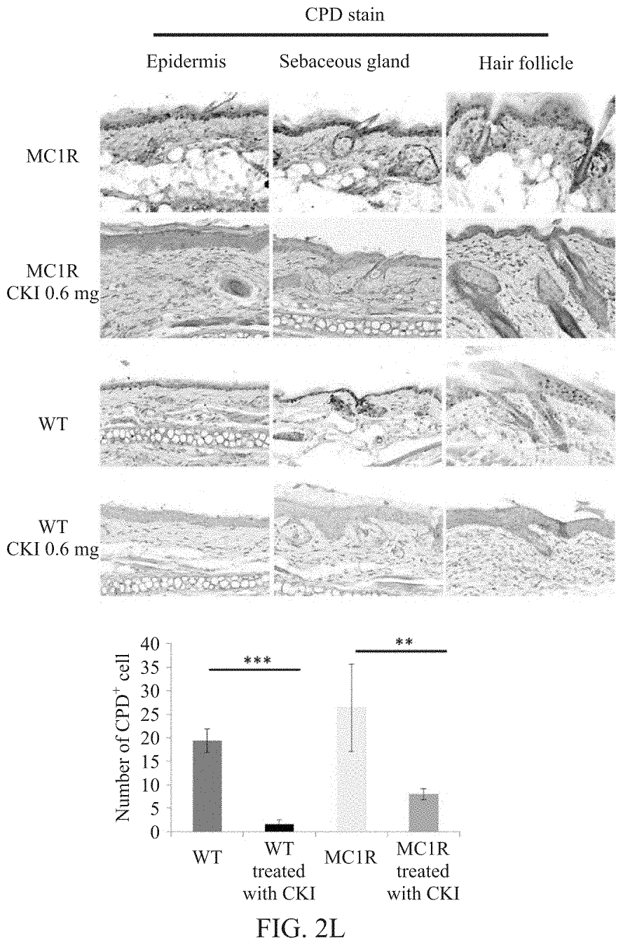

The MC1R mutant mice having a mutated MC1R gene, a deletion of a single nucleotide at position 549 that leads to 12 amino acids out-of-frame mutation, has been reported (Mountjoy, Robbins et al. 1992). To show that KitL/Kit signaling pathway is an alternative target of CKI in melanin production, the C57BL/6J-Mc1r^{e} mice were generated that carry deletion of a single nucleotide at position 549 in the MC1R gene by the CRISPR/Cas9 system. The deletion causes a frame-shift mutation and leads to loss of function of the MC1R protein, resulting in a yellow coat color in mice, due to exclusive synthesis of pheomelanin and failure to synthesize eumelanin by the melanocytes, as shown in the photograph of FIG. 2A.

As shown in FIG. 2B, C57BL/6J-Mc1r″I mouse was crossed with the K14-CreER;CK1α^{f/f} mouse to generate the K14-CreER;CK1α ^{f/f};MC1R^{KI/KI} mouse and obtain an inducible MC1R mutant mice. FIG. 2C shows the scheme used to ablate CK1α in keratinocytes of the inducible K14-CreER;CK1α ^{ff};MC1RKI¹KJ mouse.

Specifically, CK1α ablation in keratinocytes of mice at 7 weeks old was induced with intraperitoneal injection of 100 mg/kg tamoxifen for a total of 6 times on days indicated with arrows. Skin samples were harvested at days 14, 28 and 42 for analysis. FIG. 2D shows the phenotype of skin in MC1R mutant mice with CK1α ablation in keratinocytes, and it was found that pigmentation on tails was increased on day 28, compared to MC1R mutant mice without CK1α ablation in keratinocytes. In addition, Fontana-Masson staining showed increased epidermal thickness, as well as increased eumelanin pigmentation in epidermis, dermis or whole skin with a time course analysis of day 14, day 28 and day 42 after CK1α ablation in keratinocytes, as shown in FIG. 2E, and the number of Fontana-Masson-stained cells in the whole skin, epidermis and dermis was quantitated and shown in the histograms of FIG. 2F.

To investigate the effects of topical CKI application on melanin production and protection from UVB-induced DNA damage, the C57BL/6J-Mc1rmI mice received the topical CKI treatment following the scheme shown in FIG. 2G, which is the same scheme used for the wild-type mice in Example 1. As shown in FIG. 2H, Fontana-Masson staining demonstrated increased epidermal thickness and increased melanin in the epidermis after a total of 1.2 mg CKI treatment on C57BL/6J-Mcl1r^{em1} mice. Further, Western blotting analysis of the skin tissue demonstrated elevated protein levels of Kit-Ligand (KitL), c-Kit, MITF, tyrosinase and p53 in the CKI-treated group, as shown in FIG. 2I. Increased ratio of EM/PM was also found in C57BL/6J-Mc1r^{em1} mice treated with topical application of a total of CKI 2.6 mg, as shown in FIG. 2J. Additionally, after applying 750 mJ/cm² UVB irradiation on MC1R mutant mice treated with a total of 0.6 mg topical CKI, the topical CKI also reduced CPD formation in MC1R mutant mice, as shown in FIG. 2K. More CPD staining results were shown in FIG. 2L, finding that topical CKI reduces UVB-induced CPD on epidermis, sebaceous gland and hair follicle epithelial cells in MC1R mutant and wild-type mice. The results indicated that topical CKI activates p53/Kit-Ligand/c-kit pathway in MC1R-mutated mice in the same way as in the normal wild-type mice, and topical CKI treatment enhances the production of melanin and protects skin from damage of UV irradiation.

### Example 3. Topical CKI application increases melanin production and prevents DNA

damage from UVB exposure in human skin explants Different from the mouse ear skin, human skin has thicker epidermis, and melanocytes reside mainly in the epidermis. To examine the effects of topical CKI on human skin, in vitro human skin explant culture model was established using foreskin.

Following the treatment scheme as shown in FIG. 3A, with a total of 0.02 mg CKI topically applied to human skin, which is a much lower dose compared to those used on mice in Examples 1 and 2, the CKI-treated human skin explant became visibly darker in color compared to the control group treated with solvent only. FIG. 3B shows the increased ratio of EM/PM after topical CKI treatment with a total of 0.02 mg. In the immunohistochemical analysis shown in FIG. 3C, both Melan-A and Trp-1, the markers for melanocyte, were found increased in the staining. These results indicated increased melanocyte numbers in epidermis after topical CKI treatment. Furthermore, Fontana-Masson staining also showed a significantly increased melanin amount in epidermis. FIG. 3D demonstrated the Western blotting analysis results of human explant tissue, and it was found that expression levels of downstream proteins of CK1α including p53 and 0-catenin, as well as pigmentation-related proteins such as Kit-Ligand, c-kit receptor, MITF, and tyrosinase were all elevated after topical CKI treatment. In addition, human skin explants after topical CKI treatment were exposed to 1 J/cm² UVB irradiation, and collected for CPD analysis 6 hours later. The CPD analysis result showed decreased CPD levels in CKI treated group compared to the control group where only solvent is applied, as shown in FIG. 3E, indicating topical CKI treatment protects human skin from UV induced DNA damage.

### Example 4. CKI treatment on human keratinocytes and melanocytes increases in KitL production and melanocyte proliferation, maturation and migration

In skin, keratinocyte interacts with melanocyte through a system of paracrine growth factors and cell-cell adhesion junction for a stable skin homeostatic balance. In this embodiment, primary cultures of keratinocyte and melanocyte were prepared from human foreskin. The primary human keratinocytes were treated with indicated concentration of CKI for 3 days, and the conditional medium was collected and used to treat melanocytes for 3 days.

After 3 days of 10, 20 and 50 nM CKI treatment in keratinocytes, respectively, Western blotting analysis showed that expression of p53, $-catenin, KitL, and c-Kit were all dose-dependently increased, as shown in FIG. 4B. Also, the culture medium of the keratinocyte culture was then collected for ELISA analysis of KitL. It was found that KitL concentration increased both in the keratinocyte cultured medium after 50 and 75 nM CKI treatment, as shown in FIG. 4C.

Furthermore, the conditional medium derived from cultured keratinocytes was used to treat human melanocytes, and the cell behaviors of melanocytes were examined.

HMB45 is a marker of melanin transporter melanosome, and the staining with HMB45 antibody demonstrated that the intensity of melanosome, the melanocyte cell numbers, and the length of the dendrites of melanocytes were all enhanced by the CKI-treated conditional medium as shown in FIG. 4D. Western blotting analysis showed increased tyrosinase and MITF expressions in a dose-dependent manner in the melanocytes, as shown in FIG. 4E, indicating the paracrine effects of keratinocyte-derived KitL.

CKI effects on melanocytes were also examined. Various concentrations of CKI were added to the primary melanocyte culture. As shown in FIG. 4F, in the presence of CKI, the factors involved in melanocytes maturation such as MITF, tyrosinase and phosphorylated MKK6 were all increased in a dose-dependent manner. In addition, the migration ability of melanocytes was also investigated with a migration assay, and as shown in FIG. 4G, CKI treatment significantly increased the migration of melanocytes.

These results indicated that CKI treatment affects not only behavior of melanocytes through keratinocyte-derived KitL but also function of melanocytes directly.

### Example 5. UV protection effect of additional casein kinase 1 inhibitors

In addition to the CKI used in the previous embodiments, other known casein kinase 1 inhibitors such as D4476 and IC261 were also examined for their UV protection effect on mice skin, human skin explant and human keratinocytes.

As illustrated in FIG. 5A, D4476 and IC261 were topically applied on C57BL/6J-Mc1r^{e}mi mice for 2 weeks at 0.04 mg/time and 0.12 mg/week with a total of 0.24 mg applied, and the ear skin was harvested at the 3rd week for tissue analysis.

FIG. 5B shows that expressions of Kit-L, c-Kit and tyrosinase were increased after 0.24 mg treatment of D4476 and IC261, similar to the result shown in Example 2.

Topical application of D4476 on human skin explants also showed similar UV protection effect as the CKI in Example 3. As shown in the treatment scheme in FIG. 6A, human foreskins were cultured in the 12-well plate for 24 hours, and then treated with 0.04 or 0.05 mg CKI, respectively, for 2 times with a total of 0.08 or 0.1 mg of CKI applied. On the₅th day, the specimens receiving the above CKI treatments were exposed to 1,000 mJ/cm² UVB irradiation. Samples were harvested 6 hours after UV exposure for subsequent analysis.

Histology with Fontana-Masson staining of the specimens showed increased melanin intensity in epidermis treated with a total of 0.lamg of D4476, as shown in FIG. 6B. Western blotting analysis as presented in FIG. 6C and quantitated relative expression levels as presented in FIG. 6D showed stabilization of p53 and 0-catenin, similar to those shown in previous CKI in FIG. 3, and increased KitL, c-Kit, tyrosinase in specimens treated with 0.1 mg of D4476 when compared to control. Furthermore, CPD staining results in FIG. 6E showed reduced CPD in specimens treated with 0.08 mg of D4476, indicating that topical D4476 in an amount of 0.08 mg on human skin can prevent and protect from UV-induced DNA damage.

Additional casein kinase 1 inhibitors were used to examine their effects on KitL production with human keratinocyte cultures. As shown in FIG. 7A, in addition to A51, three additional casein kinase 1 inhibitors including CKI7, D4476 and IC261 at different concentrations were used to treat human keratinocytes and tested for the KitL expression levels. A51 was a CKI gifted by Dr. Yinon Ben-Neriah, and D4474 (Sigma-Aldrich D1944) and IC261 (Sigma-Aldrich 400090) were commercially available and were dissolved in DMSO. CKI7 was another CKI commercially available (Sigma-Aldrich C0742) and was dissolved in ddH₂0. All tested casein kinase 1 inhibitors showed that the expression of P-catenin was stabilized by CKI treatments. Expression of KitL was increased in CKI, CKI7, D4476 and IC261 after treatment for 72 hours, compared to DMSO or mock. Mock was a control in the experiment where the keratinocytes were neither treated with DMSO or any CKI. In FIG. 7B, it was further shown that D4476 treatment at concentrations of from 500 nM to 5,000 nM on human keratinocytes increases the expression of p53 and KitL dose-independently in treated keratinocytes after 72 hours. In FIG. 7C, it was also shown that IC261 treatment on human keratinocytes at concentrations of 500 nM to 5,000 nM increases the expressions of p53 and KitL dose-independently in treated keratinocytes after 72 hours.

These data indicated that in addition to CKI used in Examples 1 to 4, other casein kinase 1 inhibitors also have the similar ability in inducing Kit pathway and subsequent melanogenesis.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A method for preventing, ameliorating or treating a skin disorder, disease, or condition in a subject in need thereof, comprising administering an effective amount of a casein kinase 1 inhibitor to the subject.

2. The method of claim 1, wherein the skin disorder, disease, or condition is caused by UV overexposure.

3. The method of claim 2, wherein the skin disorder, disease, or condition is solar erythema, solar allergy, solar urticaria, solar elastosis, photoaging, a sunburn, an acute sunburn, a skin cancer, or any combination thereof.

4. The method of claim 1, wherein the skin disorder, disease, or condition is post-inflammatory hypopigmentation, post-wounding hyperpigmentation, actinic keratosis, atypical mole, basal cell carcinoma, melanoma, Merkel cell carcinoma, squamous cell carcinoma, cutaneous malignant melanoma, or any combination thereof.

5. The method of claim 1, wherein the skin disorder, disease, or condition is caused by a defect in a signaling pathway involving at least one of pro-opiomelanocortin (POMC), u-melanocyte stimulating hormone (u-MSH), melanocortin 1 receptor (MC1R) and microphthalmia-associated transcription factor (MITF).

6. The method of claim 1, wherein the casein kinase 1 inhibitor is topically administered to the subject.

7. A method for increasing a eumelanin level in a subject in need thereof, comprising administering an effective amount of a casein kinase 1 inhibitor to the subject for the eumelanin level to be selectively increased over a pheomelanin level in skin of the subject.

8. The method of claim 7, wherein increasing the eumelanin level increases skin pigmentation in the subject.

9. The method of claim 8, wherein the skin pigmentation protects the subject from ultraviolet radiation.

10. The method of claim 7, wherein the eumelanin level is increased in epidermis of the subject.

11. The method of claim 7, wherein increasing the eumelanin level involves an increase in a KitL level in epidermis of the subject.

12. The method of claim 11, wherein increasing the KitL level in the epidermis induces movement of melanocytes from dermis to the epidermis in the subject.

13. The method of claim 7, wherein the casein kinase 1 inhibitor is administered topically to the subject.

14. The method of claim 7, wherein the subject is a human.

15. A method for inhibiting activity of a casein kinase 1 in a skin cell, comprising contacting the skin cell with an effective amount of a casein kinase 1 inhibitor.

16. The method of claim 15, wherein contacting the skin cell with the effective amount of the casein kinase 1 inhibitor increases a eumelanin level in the skin cell.

17. The method of claim 15, wherein the skin cell is an epidermal cell.

18. The method of claim 15, wherein the casein kinase 1 inhibitor is a casein kinase la inhibitor.

19. The method of claim 15, wherein the casein kinase 1 inhibitor is selected from the group consisting of CKI7, D4476, IC261, and a compound represented by formulas I to VII: 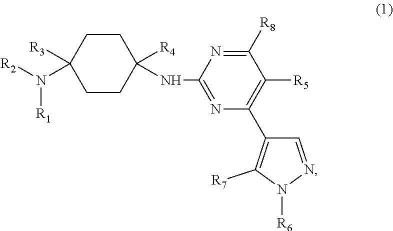
wherein:
R₁ and R₂ are each independently selected from the group consisting of H, straight or branched C1-C8 alkyl, straight or branched C1-C5 alkoxy, straight or branched C1-C5 acyl, C5-C15 aryl, and C3-C7 heteroaryl each optionally substituted by at least one of halide, hydroxyl, ester, ether, C5-C15 aryl, C3-C7 heteroaryl, and amide; or R₁ and R₂ together with the nitrogen atom they are connected to form a 4-7 membered saturated, unsaturated or aromatic ring optionally including at least one of N, 0, NH, C=N, C═O and SO2 and optionally substituted with at least one of straight or branched C1-C5 alkyl, C5-C15 aryl, C3-C7 heteroaryl, hydroxyl, halide and cyano;
R3 and R₄ are each independently selected from the group consisting of H, straight or branched C1-C8 alkyl optionally substituted by at least one of halide, hydroxyl, alkoxy, C5-C15 aryl, C3-C7 heteroaryl, ester and amide; or
R₁ or R₂ together with R₃ and the carbon and nitrogen atom they are each connected to form a 4-7 membered saturated, unsaturated or aromatic ring optionally including at least one of N, NH, 0, C=N, C=0, and S02, and optionally substituted with at least one of straight or branched C1-C5 alkyl, C5-C15 aryl, C3-C7 heteroaryl, hydroxyl, carbonyl, and halide;
R₅ and Rs are each independently selected from the group consisting of H, halide, straight or branched C1-C8 alkyl, straight or branched C2-C8 alkenyl, and straight or branched C2-C8 alkynyl optionally substituted by at least one halide;
R₆ is selected from the group consisting of straight or branched C1-C8 alkyl, Straight or branched C2-C8 alkenyl, straight or branched C2-C8 alkynyl, C5-C10 cycloalkyl, and saturated or unsaturated 4-6 membered heterocycle optionally substituted by at least one of straight or branched C1-C8 alkyl, C3-C7 cycloalkyl, 4-6 membered heterocycle, C5-C15 aryl, C3-C7 heteroaryl, halide, hydroxyl, and C1-C5 alkyl halide;
R₇ is selected from the group consisting of straight or branched C1-C8 alkyl, straight or branched C2-C8 alkenyl, and straight or branched C2-C8 alkynyl optionally substituted by at least one of C3-C7 cycloalkyl, 4-6 membered heterocycle, C5-C15 aryl, C3-C7 heteroaryl, halide, hydroxyl, and C1-C5 alkyl halide; [chemical structure]
